(19) United States
(12) Patent Application Publication
(10) Pub. No.: US 2023/0129413 A1
(43) Pub. Date: Apr. 27, 2023

(54) Levodopa and Carbidopa Intestinal Gel and Methods of Use

(71) Applicant: AbbVie Inc., North Chicago, IL (US)

(72) Inventors: Alexandru DEAC, Skokie, IL (US); Ye HUANG, Gurnee, IL (US); John M. LIPARI, Mount Pleasant, WI (US); Alexander RUGGLES, Lake Forest, IL (US); Thin Yu TUN, Gurnee, IL (US)

(73) Assignee: AbbVie Inc., North Chicago, IL (US)

(21) Appl. No.: 17/817,191

(22) Filed: Aug. 3, 2022

Related U.S. Application Data
(63) Continuation of application No. 16/854,079, filed on Apr. 21, 2020, now abandoned.
Application No. 16/854,079 is a continuation of application No. 15/655,078, filed on Jul. 20, 2017, now abandoned.
(60) Provisional application No. 62/364,770, filed on Jul. 20, 2016.

(51) Int. Cl. A61K 31/198; A61K 47/32; A61K 47/10; A61K 47/38; A61K 9/10; A61K 9/00; A61K 31/195

(52) U.S. Cl. CPC A61K 31/198; A61K 47/32; A61K 47/10; A61K 47/38; A61K 9/10; A61K 9/0053; A61K 9/00; A61K 31/195

(57) ABSTRACT

The present disclosure provides (a) a pharmaceutical composition comprising a levodopa active agent and a carbidopa active agent and (b) methods of treating Parkinson's disease and associated conditions comprising administering the pharmaceutical composition to a subject with Parkinson's disease.

### FIELD OF THE INVENTION

The present disclosure provides (a) a pharmaceutical composition comprising a levodopa active agent and a carbidopa active agent; and (b) methods of treating Parkinson's disease and associated conditions comprising administering the pharmaceutical composition to a subject with Parkinson's disease.

### BACKGROUND OF THE INVENTION

Parkinson's disease is a chronic and progressive neurodegenerative condition characterized by reduced levels in the brain of the neurotransmitter dopamine (i.e., 3,4-dihydroxyphenethylamine). Administration of levodopa (or L-dopa) currently is the most effective therapy for treating a patient with Parkinson's disease. L-dopa, which unlike dopamine can cross the blood-brain barrier, is enzymatically converted in the brain to dopamine resulting in an increase in dopamine levels:

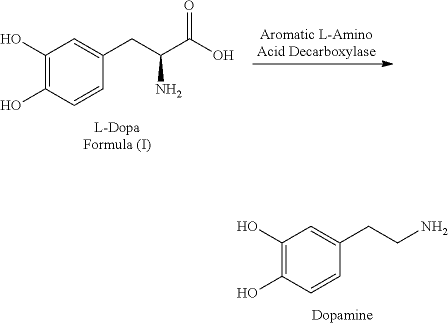 [SMILES: N[C@@H](Cc1ccc(O)c(O)c1)C(=O)O ; NCCc1ccc(O)c(O)c1]

The conversion of L-dopa to dopamine is catalyzed by aromatic L-amino acid decarboxylase, a ubiquitous enzyme that promotes central as well as peripheral metabolism of L-dopa to dopamine. A relatively large dose of L-dopa is required to achieve therapeutically effective dopamine levels in the brain. Administration of such large L-dopa doses results in elevated peripheral dopamine levels that can cause nausea in some patients. To overcome these problems, L-dopa generally is co-administered with a peripheral aromatic L-amino acid decarboxylase inhibitor such as carbidopa (i.e., (2S)-3-(3,4-dihydroxy-phenyl)-2-hydrazino methylpropanoic acid):

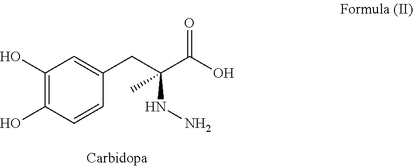 [SMILES: C[C@@](Cc1ccc(O)c(O)c1)(NN)C(=O)O]

Co-administration of carbidopa with L-dopa inhibits the peripheral metabolism of L-dopa to dopamine, which significantly reduces the L-dopa dose required for a therapeutically effective response and reduces the associated side effects.

Even when L-dopa and carbidopa are co-administered, however, it is difficult to consistently maintain the desired dopamine levels in the brain due to the relatively short half-life of L-dopa in plasma. In addition, the tolerance of many patients to variability in dopamine levels in the brain decreases as the disease progresses. One approach that has been effective in reducing variability of dopamine levels is the continuous intestinal delivery of an adjustable dose of an L-dopa/carbidopa gel known by its commercial name, DuoDopa®. DuoDopa® is a suspension of L-dopa/carbidopa monohydrate (4:1 ratio of L-dopa to carbidopa monohydrate) in an aqueous gel. The gel is delivered to the proximal small intestine through a jejunal tube inserted through a percutaneous endoscopic gastrostomy port. DuoDopa® is packaged in disposable drug reservoirs (“DDRs”) and continuously administered via a software-controlled ambulatory infusion pump. Although L-dopa and carbidopa have been co-administered to treat Parkinson's disease for several decades, a pharmaceutical gel composition suitable for storage at room temperature is not currently commercially available.

The current composition of the DuoDopa® L-dopa/carbidopa intestinal gel is a gel for continuous intestinal administration. For long-term administration, the gel is administered with a portable pump directly into the duodenum or upper jejunum via a percutaneous endoscopic gastrostomy tube with an inner intestinal/jejunal tube. Each 1 ml of Duodopa® contains 20 mg levodopa and 5 mg carbidopa monohydrate. Despite the current commercial success of DuoDopa®, the product is subject to limitations in product preparation, including (1) risk of sedimentation of drug particles during storage and administration, (2) chemical instability of carbidopa, which leads to hydrazine formation.

Accordingly, there is a continuing need for improved formulations and methods that can provide continuous and consistent dopamine levels in the brain to effectively treat movement disorders such as Parkinson's disease. The present disclosure provides such improved formulations and methods.

### SUMMARY OF THE INVENTION

In one aspect, the present disclosure provides a pharmaceutical composition comprising a levodopa active agent and a carbidopa active agent for intraduodenal administration, wherein the levodopa active agent and the carbidopa active agent are suspended in one or more polymer-based suspending agents, e.g., a carbomer, and the pharmaceutical composition has a yield value of at least about 0.3 Pascal (Pa) and an acceptance value of ≤15. The levodopa active agent may be provided in an amount of about 4 weight/weight percent (w/w %) of the composition and the carbidopa active agent (e.g., carbidopa monohydrate) may be provided in an amount of about 1 weight/weight percent of the composition. Pharmaceutical compositions of the disclosure have a yield value that provides physical stability for at least about 8 weeks, at least about 15 weeks, at least about 26 weeks, or at least about 60 weeks, under one or more storage conditions, e.g., at 5° C., at room temperature (e.g., about 20° C. to about 25° C.) and a relative humidity (RH) of about 60%, or at about 40° C. and a RH of about 75%.

In another aspect, the present disclosure provides a pharmaceutical composition comprising a levodopa active agent and a carbidopa active agent for intraduodenal administration wherein the levodopa active agent and the carbidopa active agent are suspended in one or more polymer-based suspending agents and the pharmaceutical composition has a yield value of at least about 1.1 Pascal (Pa). The levodopa active agent may be provided in an amount of about 4 weight/weight percent (w/w %) of the composition and the carbidopa active agent (e.g., carbidopa monohydrate) may be provided in an amount of about 1 weight/weight percent of the composition. The pharmaceutical composition has a desired yield value suitable for physical stability for at least about 15 weeks at room temperature (e.g., about 20° C. to about 25° C.).

In another aspect, the present disclosure provides a pharmaceutical composition comprising a levodopa active agent and a carbidopa active agent for intraduodenal administration wherein the levodopa active agent is provided in an amount of about 4 weight/weight percent (w/w %) of the composition and carbidopa (e.g., carbidopa monohydrate) is provided in an amount of about 1 weight/weight percent of the composition wherein the levodopa and carbidopa are suspended in an aqueous carrier and is stored in individual disposable drug reservoirs (“DDR”). The DDR containing the pharmaceutical composition is covered by a material that acts as a complete oxygen barrier, such as a foil pouch. This packaging ensures that the pharmaceutical composition is suitable for chemical stability for at least about 15 weeks at room temperature (e.g., about 20° C. to about 25° C.).

In another aspect, the present disclosure provides a method of treating Parkinson's disease in a patient in need thereof, wherein the method comprises administering to the patient a pharmaceutical composition comprising a levodopa active agent and a carbidopa active agent for intraduodenal administration wherein the levodopa active agent and the carbidopa active agent are suspended in one or more polymer-based suspending agents and the pharmaceutical composition has a yield value of at least about 4 Pa. The levodopa agent may be provided in an amount of about 4 weight/weight percent (w/w %) of the composition and the carbidopa active agent (e.g., carbidopa monohydrate) may be provided in an amount of about 1 weight/weight percent of the composition. The pharmaceutical composition has a desired yield value suitable for physical stability for at least about 15 weeks at room temperature (e.g., about 20° C. to about 25° C.).

In another aspect, the present disclosure relates to methods of manufacturing a pharmaceutical composition of the disclosure, in particular a high concentration pharmaceutical composition as disclosed, for example, in Example 1 and FIG. 1 below.

These and additional embodiments of the disclosure are further described herein.

Further benefits of the present disclosure will be apparent to one skilled in the art from reading this patent application. The embodiments of the disclosure described in the following paragraphs are intended to illustrate the invention and should not be deemed to narrow the scope of the invention.

### BRIEF DESCRIPTION OF THE DRAWINGS

FIG. 1 is a manufacturing process flowchart for producing an exemplary pharmaceutical formulation of the disclosure.

FIG. 2 is a line graph showing the L-Dopa blood level time-concentration profile in mini-pigs of an exemplary pharmaceutical composition of the disclosure as against two comparators, all given as a bolus plus a six-hour continuous infusion.

FIG. 3 is a line graph showing the carbidopa blood level time-concentration profile in mini-pigs of an exemplary pharmaceutical composition of the disclosure as against two comparators, all given as a bolus plus in a six-hour continuous infusion.

FIG. 4A is a line graph showing the dissolution rate of levodopa in a pH 4.5 media for a levodopa carbidopa intestinal gel made with Carbopol® 974P and packaged with a foil pouch at initial time point and 15 weeks.

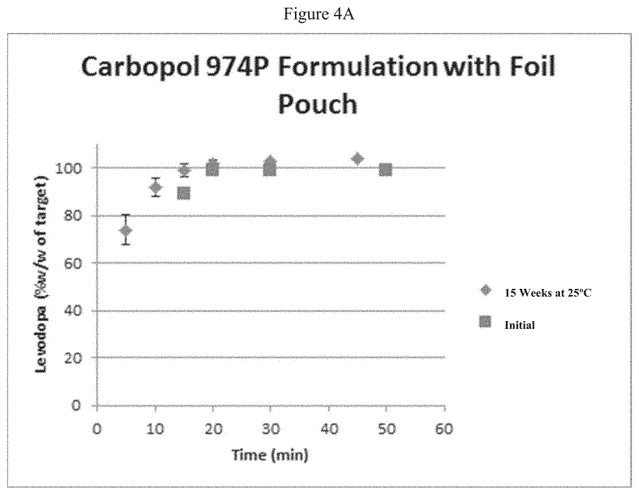

FIG. 4B is a line graph showing the dissolution rate of carbidopa in a pH 4.5 media for a levodopa carbidopa intestinal gel made with Carbopol® 974P and packaged with a foil pouch at initial time point and 15 weeks.

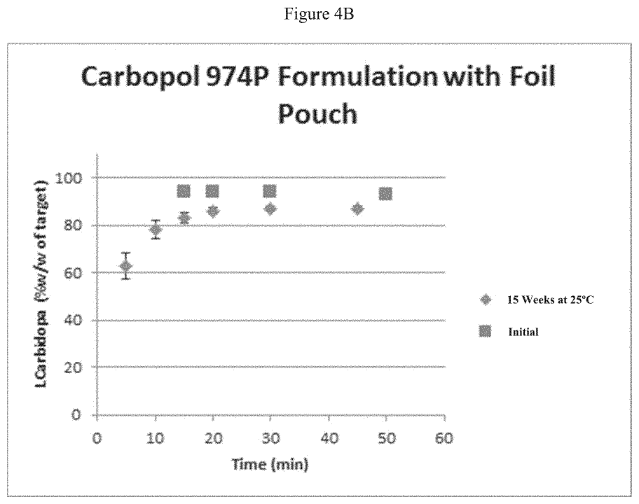

FIG. 5 is an illustration showing 3 different vials filled with levodopa-carbidopa intestinal gel made with Carbopol® 971P. The samples, from left to right, have the following 971P concentrations: 0.5% w/w, 0.2% w/w, 0.1% w/w (or w/w %).

FIG. 6 is a line graph showing low shear (LS) viscosity of pharmaceutical compositions comprising levodopa and carbidopa after storage at various conditions.

FIG. 7 is a line graph showing high shear (HS) viscosity of pharmaceutical compositions comprising levodopa and carbidopa after storage at various conditions.

FIG. 8 is a diagram showing the process of how the acceptance value of a pharmaceutical composition is determined.

FIG. 9 is a line graph showing the pharmacokinetics of Formulation B (large levodopa particle size) and Formulation C (small levodopa particle size) in mini-pigs.

### DETAILED DESCRIPTION OF THE INVENTION

This written description uses examples to disclose the invention, including the best mode, and also to enable any person skilled in the art to practice the invention, including making and using any of the disclosed pharmaceutical compositions, kits, pharmaceutical dosage forms, and performing any of the disclosed methods or processes. The patentable scope of the invention is defined by the claims, and may include other examples that occur to those skilled in the art. Such other examples are intended to be within the scope of the claims if they have elements that do not differ from the literal language of the claims, or if they include equivalent elements.

### I. Definitions

Section headings as used in this section and the entire disclosure are not intended to be limiting.

Where a numeric range is recited, each intervening number within the range is explicitly contemplated with the same degree of precision. For example, for the range 6 to 9, the numbers 7 and 8 are contemplated in addition to 6 and 9, and for the range 6.0 to 7.0, the numbers 6.0, 6.1, 6.2, 6.3, 6.4, 6.5, 6.6, 6.7, 6.8, 6.9 and 7.0 are explicitly contemplated. In the same manner, all recited ratios also include all sub-ratios falling within the broader ratio.

The singular forms “a,” “an,” and “the” include plural referents unless the context clearly dictates otherwise.

The term “and/or” as used in a phrase such as “A and/or B” herein is intended to include “A and B”, “A or B”, “A”, and “B”.

The term “about” generally refers to a range of numbers that one of skill in the art would consider equivalent to the recited value (i.e., having the same function or result). In many instances, the term “about” may include numbers that are rounded to the nearest significant figure.

In some embodiments, the term “about” includes the recited number±10%. Thus, “about 10” means 9 to 11.

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below.

The terms “improve” and “improving” have their plain and ordinary meaning to one skilled in the art of pharmaceutical or medical sciences and specifically include ameliorating the effects of Parkinson's disease, or decreasing or lessening a symptom or side effect of Parkinson's disease.

The term “patient” includes mammals and humans, particularly humans.

The term “pharmaceutically acceptable excipient” refers to any and all solvents, dispersion media, preservatives, antioxidants, isotonic and absorption delaying agents, and the like, that are compatible with pharmaceutical administration. Dispersion media may include water insoluble excipients such as microcrystalline cellulose.

The term “pharmaceutically acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methyl-bicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

The terms “reduce” and “reducing” have their plain and ordinary meanings to one skilled in the art of pharmaceutical or medical sciences and specifically include diminishing or decreasing the number of occurrences, the duration, or the intensity, of a Parkinson's disease symptom or side effect, such as dyskinesias or hallucinations.

The term “therapeutically effective amount” means an amount of a compound that, when administered to a patient suffering from or susceptible to Parkinson's disease or an associated condition is sufficient, either alone or in combination with additional therapies, to effect treatment for Parkinson's disease or the associated condition. The “therapeutically effective amount” will vary depending, for example, on the compound, pharmaceutical composition or pharmaceutical dosage form, the condition treated and its severity, and the age and weight of the patient to be treated.

The terms “treat” and “treating” have their plain and ordinary meaning to one skilled in the art of pharmaceutical or medical sciences and specifically include improving the quality of life or reducing the symptoms or side effects of Parkinson's disease.

The terms “a” and “an” may refer to one or more than one.

The term “levodopa active agent” as used herein refers to levodopa, and pharmaceutically acceptable salts or hydrates thereof. In one embodiment, the levodopa active agent is a hydrated form of levodopa. In another embodiment, the levodopa active agent is levodopa monohydrate. In another embodiment, the levodopa active agent is levodopa. Levodopa is also referred to as L-dopa.

The term “carbidopa active agent” as used herein refers to carbidopa, and pharmaceutically acceptable salts or hydrates thereof. In one embodiment, the carbidopa active agent is a hydrated form of carbidopa. In another embodiment, the carbidopa active agent is carbidopa monohydrate. In another embodiment, the carbidopa active agent is carbidopa.

The term “room temperature” generally refers to ambient conditions at a temperature of about 20 to about 25 degrees Celsius.

### II. Pharmaceutical Compositions

As discussed above, pharmaceutical compositions comprising a levodopa and carbidopa suspension for intraduodenal administration face various challenges including sedimentation of drug particles during storage and administration and lack of chemical stability during storage. In order to address these challenges, it has been unexpectedly discovered that such challenges may be overcome when the pharmaceutical composition has a suitable yield value and is packaged in a foil pouch or any other packaging that acts as an oxygen barrier. Yield value relates to the initial resistance to flow under stress of a fluid; thus, yield value may also be referred to as yield stress. Yield value affects the suspending ability of a medium (e.g., vehicle and/or suspending agent. Specifically, particles dispersed in a medium can remain suspended if the yield value of the medium is sufficient to overcome the effect of gravity or buoyancy on those particles. Yield value is independent of viscosity.

Thus, the present disclosure provides a pharmaceutical composition comprising a levodopa active agent and a carbidopa active agent for intraduodenal administration, wherein the levodopa active agent and the carbidopa active agent are present in a therapeutically effective amount suspended in one or more polymer-based suspending agents and wherein the pharmaceutical composition has a suitable yield value. Examples of suitable yield values include, but are not limited to, at least about 1.1 Pa. In another embodiment, examples of suitable yield values include, but are not limited to, at least about 0.3 Pa.

As understood herein, yield value is measured using a Discovery HR-2 rheometer from TA instruments. The rheometer can be fitted with a concentric cylinder, or other suitable geometry (e.g. cone and plate). The sample is first soaked at 25° C. for 600 seconds, or until it reaches equilibrium. The sample may be presheared at 200 l/s for 120 seconds if needed. An ascending flow-ramp is performed by first soaking the presheared sample for 360 seconds, then increasing the shear rate from 0.001 l/s to 250 l/s over 300 seconds, or other suitable range that can give a good resolution of the yield value. The sample can then held at 250 l/s for an additional 300 seconds, if needed. Finally, a descending flow-ramp is performed from 250 l/s to 0.001 l/s over 300 seconds, or other suitable range that can give a good resolution of the yield value. The resulting descending or ascending flow-ramp are fitted with the Herschel-Bulkley model to obtain the yield value.

The pharmaceutical compositions described herein having the above-described yield value resist sedimentation at room temperature (e.g., about 20° C. to about 25° C., such as about 22° C.) for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks.

In another embodiment, the pharmaceutical compositions described herein having a yield value of at least about 0.3 Pa are physically stable and resist sedimentation at room temperature when stored for at least about 8 weeks, at least about 10 weeks, at least about 15 weeks, at least about 20 weeks, at least about 26 weeks, at least about 30 weeks, at least about 40 weeks, at least about 50 weeks, or at least about 60 weeks.

In another embodiment, when packaged in a foil pouch or, other suitable packaging that acts as an oxygen barrier, the pharmaceutical compositions are chemically stable at room temperature (e.g., about 20° C. to about 25° C., such as about 22° C.) for at least about 15 weeks. Additionally, the pharmaceutical compositions described herein are packaged in a foil pouch, or other suitable packaging that acts as an oxygen barrier, are stable at room temperature (e.g., about 20° C. to about 25° C., such as about 22° C.) for at least about 15 weeks, or alternatively for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks.

In another embodiment, when packaged in a foil pouch or, other suitable packaging that acts as an oxygen barrier and, optionally, when either the pharmaceutical composition or foil pouch comprises an oxygen scavenger, the pharmaceutical compositions are chemically stable at room temperature (e.g., about 20° C. to about 25° C., such as about 22° C.) for at least about 8 weeks. In another embodiment, the pharmaceutical compositions are chemically stable at room temperature for at least about 9 weeks, at least about 10 weeks, at least about 11 weeks, at least about 12 weeks, at least about 13 weeks, at least about 14 weeks, at least about 15 weeks, at least about 26 weeks, or at least about 60 weeks. Oxygen scavengers include, but are not limited to a mixture of iron powder and sodium chloride, activated carbon, and ascorbic acid.

In various aspects, the therapeutically effective amount of a levodopa active agent and a carbidopa active agent (e.g., carbidopa monohydrate) present in the pharmaceutical composition may be about 4.0 and 1.0 weight/weight percent of the composition, respectively.

In one embodiment, the pharmaceutical composition comprises a levodopa active agent in an amount of about 4.0 weight/weight percent of the total composition; a carbidopa active agent (e.g., carbidopa monohydrate) in an amount of about 1.0 weight/weight percent of the total composition; at least one suspending agent (e.g., polymer-based); and a liquid vehicle (for example, water, polyethylene glycol). In various embodiments, the liquid vehicle can make up from about zero weight/weight percent to about 95 weight/weight percent of the total composition, for example from about 10 weight/weight percent to about 70 weight/weight percent, or from about 40 weight/weight percent to about 60 weight/weight percent of the total composition.

In one embodiment, the levodopa active agent is levodopa and pharmaceutically acceptable salts or hydrates thereof, such as levodopa monohydrate. In one embodiment, levodopa is present in the composition in an amount of from about 1.0 to 5.0 weight/weight percent in the total composition. In one embodiment the pharmaceutical composition comprises about 4.0 weight/weight percent of a levodopa active agent. In one embodiment, the levodopa active agent can be processed into microparticles or microspheres or the like, for example as described in Example 1 below, for inclusion in the present pharmaceutical compositions.

In one embodiment, the carbidopa active agent is carbidopa and pharmaceutically acceptable salts or hydrates thereof, such as carbidopa monohydrate. In one embodiment, the carbidopa active agent is present in the composition in an amount of from about 0.25 to 1.25 weight/weight percent in the total composition. In one embodiment the pharmaceutical composition comprises about 1.0 weight/weight percent of a carbidopa active agent. In one embodiment, the form of carbidopa active agent to be administered is a hydrated form of carbidopa. In one embodiment, the form of carbidopa active agent to be administered is carbidopa monohydrate. In one embodiment, the carbidopa active agent can be processed into microparticles or microspheres or the like, for example as described in Example 1 below, for inclusion in the present pharmaceutical compositions.

The levodopa active agent and carbidopa active agent may be present in the pharmaceutical composition in any suitable ratio, for example, the ratio of levodopa active agent to carbidopa active agent (e.g., carbidopa monohydrate) in the present pharmaceutical compositions may be about 4:1. For example, the pharmaceutical composition can comprise about 4 weight/weight percent of levodopa active agent and 1 weight/weight percent carbidopa active agent (e.g., carbidopa monohydrate). In one embodiment, the pharmaceutical composition comprises a liquid or viscous liquid comprising about 200 mg levodopa and about 50 mg carbidopa (e.g., carbidopa monohydrate) per each 5.0 mL volume. In one embodiment, the levodopa active agent and the carbidopa active agent are processed into microparticles or microspheres or the like, for example as described in Example 1 below, for inclusion in the present pharmaceutical compositions.

In another embodiment, a suitable suspending agent can be any polymer-based suspending agent that provides a pharmaceutical composition having a yield value of at least about 0.3 Pa. Pharmaceutical compositions of the disclosure may comprise one or more suspending agents. Examples of suitable polymer-based suspending agents include, but are not limited to, acrylic acid-based polymers, e.g., polymers primarily made from acrylic acid known as carbomer or acrylic acid-based polymers sold under the trade name Carbopol®, and hydrocolloid polymers, e.g., locust beam gum, guar gum, methylcellulose, sodium carboxymethylcellulose with microcrystalline cellulose, e.g., Avicel® CL-611 or Avicel® RC 591, xanthan gum, e.g., Vanzan, and gum tragacanth. Carbomer can be a high molecular weight polymer of acrylic acid crosslinked with ally ethers of polyalcohols. Acrylic acid-based polymers may be cross-linked, for example, cross-linked with polyalkenyl ethers or divinyl glycol. In particular, the one or more suspending agents may be Carbopol® 934P, Carbopol® 971P or Carbopol® 974P. Carbopol® 934P can be described by the USP/NF Pharmacopeia Monograph Compendial Name Carbomer 934P. Carbopol® 971P can be described by the USP/NF Pharmacopeia Monograph Compendial Name Carbomer Homopolymer Type A. Carbopol® 974P can be described by the USP/NF Pharmacopeia Monograph Compendial Name Carbomer Homopolymer Type B. Polymer-based suspending agents, such as Carbopol® 971P and Carbopol® 974P, are synthetic, which reduces lot-to-lot variability, unlike the cellulose-based agents. Furthermore, polymer-based suspending agents, such as Carbopol® 971P and Carbopol® 974P, do not substantially dissolve thereby reducing any negative interaction with the levodopa active agent and carbidopa active agent. Also, polymer-based suspending agents, such as Carbopol® 971P and Carbopol® 974P, are not cellulose-based, which reduces the environmental impact of producing such cellulose-based agents. The one or more suspending agents are incorporated into the composition in an amount of from about 0.1 to about 6 w/w % of the total weight of the pharmaceutical composition. The one or more suspending agents can be used in any amount within the range, including for example, about 0.1 to about 1 w/w %, about 0.1 to about 0.5 w/w %, or about 0.1 to about 0.2 w/w %.

In another embodiment, a suitable suspending agent can be any polymer-based suspending agent that provides a pharmaceutical composition having a yield value of at least about 4 Pa. Examples of suitable polymer-based suspending agents include, but are not limited to, acrylic acid-based polymers, e.g., polymers primarily made from acrylic acid known as carbomer or acrylic acid-based polymers sold under the trade name Carbopol®, and hydrocolloid polymer, e.g., locust beam gum, guar gum, methylcellulose, sodium carboxymethylcellulose with microcrystalline cellulose and gum tragacanth. Acrylic acid based polymers may be cross-linked, for example, cross-linked with polyalkenyl ethers or divinyl glycol. In particular, the one or more suspending agent may be Carbopol® 971P and Carbopol® 974P. Polymer-based suspending agents, such as Carbopol® 971P and Carbopol® 974P, are synthetic, which reduces lot-to-lot variability, unlike the cellulose-based agents. Furthermore, polymer-based suspending agents, such as Carbopol® 971P and Carbopol® 974P, do not substantially dissolve thereby reducing any negative interaction with the levodopa active agent and carbidopa active agent. Also, polymer-based suspending agents, such as Carbopol® 971P and Carbopol® 974P, are not cellulose-based, which reduces the environmental impact of producing such cellulose-based agents. The suspending agent is incorporated into the composition in an amount of from about 0.1 to about 5 w/w % of the total weight of the pharmaceutical composition. The suspending agent can be used in any amount within the range, including for example, about 0.1 to about 1 w/w %, or about 0.1 to 0.5 w/w %.

In another embodiment, the suspending agent is an acrylic acid-based polymer, e.g., a carbomer, e.g., Carbopol® 971P or Carbopol® 974P, and the composition comprises the acrylic acid-based polymer in an amount of about 0.1 w/w % to about 0.5 w/w % of the total composition, e.g., in an amount of about 0.1 w/w % to about 0.3 w/w % of the total composition, e.g., in an amount of about 0.1 w/w % to about 0.2 w/w % of the total composition. In another embodiment, the composition comprises the acrylic acid-based polymer in an amount of about 0.10 w/w % of the total composition. In another embodiment, the composition comprises the acrylic acid-based polymer in an amount of about 0.11 w/w %, about 0.12 w/w %, about 0.13 w/w %, about 0.14 w/w %, about 0.15 w/w %, about 0.16 w/w %, about 0.17 w/w %, about 0.18 w/w %, about 0.19 w/w %, about 0.20 w/w %, or about 0.20 w/w %, of the total composition.

In another embodiment, the suspending agent is a hydrocolloid polymer, e.g., locust beam gum, guar gum, methylcellulose, sodium carboxymethylcellulose with microcrystalline cellulose, or gum tragacanth, and the composition comprises the hydrocolloid polymer in an amount of about 1 w/w % to about 6 w/w % of the total composition. In another embodiment, the composition comprises the hydrocolloid polymer in an amount of about 1 w/w % of the total composition. In another embodiment, the composition comprises the hydrocolloid polymer in an amount of about 2 w/w %, about 3 w/w %, about 4 w/w %, or about 5 w/w % of the total composition

For the present compositions, one or more suspending agents can be used to obtain a suspension for intraduodenal administration having the desired yield value as set forth above.

However, when a surfactant is used, it may be best to add the surfactant or surfactants following addition of levodopa active agent and carbidopa active agent and suspending agent as taught herein.

It should be understood that each component comprising the compositions of the present disclosure must be pharmaceutically acceptable and safe for administration in a formulation intended to be administered in human subjects.

An antimicrobial agent can be added to preserve the composition by inhibiting microbial growth. Any pharmaceutically acceptable antimicrobial agent can be used for example, such as, sodium benzoate, sodium propionate, and sorbates. The surfactant or surfactants can be incorporated following the addition of levodopa active agent and carbidopa active agent and suspending agent as taught herein.

When a carbomer, i.e. Carbopol® 971P or Carbopol® 974P, is used as the suspending agent, a neutralizing agent must be added. Examples of neutralizing agents are, but not limited to, inorganic bases, e.g. sodium hydroxide and potassium hydroxide, or amines, e.g. triethanolamine and tromethamine.

In one embodiment, the pharmaceutical composition is a viscous liquid composition. In one aspect, the pharmaceutical composition comprises water and is suitable for infusion.

In another embodiment, the pharmaceutical composition has a levodopa active agent concentration of at least about 5 mg/mL. In one aspect, the levodopa active agent concentration is at least about 10 mg/mL. In another aspect, the levodopa active agent concentration is at least about 20 mg/mL. In another aspect, the levodopa active agent concentration is at least about 30 mg/mL. In another aspect, the levodopa active agent concentration is at least about 35 mg/mL. In another aspect, the levodopa active agent concentration is at least about 40 mg/mL. In another aspect, the levodopa active agent concentration is at least about 45 mg/mL. In another aspect, the levodopa active agent concentration is at least about 50 mg/mL. In another aspect, the levodopa active agent concentration is at least about 100 mg/mL. In another aspect, the levodopa active agent concentration is at least about 150 mg/mL. In another aspect, the levodopa active agent concentration is at least about 200 mg/mL.

In another embodiment, the pharmaceutical composition has a carbidopa active agent (e.g., carbidopa monohydrate) concentration of at least about 5 mg/mL. In one aspect, the carbidopa active agent concentration is at least about 10 mg/mL. In another aspect, the carbidopa active agent concentration is at least about 20 mg/mL. In another aspect, the carbidopa active agent concentration is at least about 30 mg/mL. In another aspect, the carbidopa active agent concentration is at least about 50 mg/mL. In another aspect, the carbidopa active agent concentration is at least about 100 mg/mL. In another aspect, the carbidopa active agent concentration is at least about 150 mg/mL. In another aspect, the active agent carbidopa concentration is at least about 200 mg/mL.

The pharmaceutical compositions of the present disclosure optionally comprise one or more additional pharmaceutically acceptable excipients. The term “excipient” refers to any substance, not itself a therapeutic agent, used as a carrier or vehicle for delivery of a therapeutic agent to a subject or added to a pharmaceutical composition to improve its handling or storage properties or to permit or facilitate formation of a unit dose of the composition.

Excipients include, for example, antioxidants, agents to adjust the pH and osmolarity, preservatives, thickening agents, colorants, buffering agents, bacteriostats, and stabilizers. A given excipient, if present, generally will be present in an amount of about 0.001% to about 95%, about 0.01% to about 80%, about 0.02% to about 25%, or about 0.3% to about 10%, w/w %.

In one embodiment, the pharmaceutical compositions optionally comprise an antioxidant. Suitable antioxidants for use in the pharmaceutical compositions include, for example, butylated hydroxytoluene, butylated hydroxyanisole, potassium metabisulfite, cysteine, and the like.

In one embodiment, the pharmaceutical compositions optionally comprise a buffering agent. Buffering agents include agents that reduce pH changes. Suitable classes of buffering agents for use in various embodiments of the present disclosure comprise a salt of a Group IA metal including, for example, a bicarbonate salt of a Group IA metal, a carbonate salt of a Group IA metal, an alkaline or alkali earth metal buffering agent, an aluminum buffering agent, a calcium buffering agent, a sodium buffering agent, or a magnesium buffering agent. Suitable buffering agents further include carbonates, phosphates, bicarbonates, citrates, borates, acetates, phthalates, tartrates, succinates of any of the foregoing, for example, sodium or potassium phosphate, citrate, borate, acetate, bicarbonate and carbonate.

In one embodiment, the composition has a pH from about 3.5 to about 8. In one aspect, the pH is from about 3.5 to about 7.5. In another aspect, the pH is from about 4.0 to about 7.5. In another aspect, the pH is from about 5.0 to about 7.5. In another aspect, the pH is from about 5.5 to about 7.5. In another aspect, the pH is from about 6.0 to about 7.5.

In various embodiments, the pharmaceutical composition may be present in a container. Suitable containers include containers (e.g., a bag) which are oxygen impermeable. These oxygen permeability barriers may be incorporated into the primary container or a secondary outer container. Non-limiting examples of suitable containers include foil pouches.

In another embodiment, a pharmaceutical dosage form is provided. The pharmaceutical dosage form may comprise the pharmaceutical composition described herein in a DDR having an oxygen impermeable enclosure disposed therein, wherein the oxygen impermeable enclosure is purged with an inert gas (e.g., N₂). An oxygen scavenger (e.g., ferrous or non-ferrous based, canister or sachet) may also be added. The pharmaceutical dosage form may suitable for use in a continuous infusion pump capable of delivering the composition in a therapeutically effective manner. A suitable oxygen impermeable enclosure can include, for example, a foil bag.

### III. Methods of Preparing a Pharmaceutical Composition

The present disclosure further provides methods of preparing the pharmaceutical compositions described herein. In various aspects, the methods of preparing the pharmaceutical composition described herein can comprise providing a levodopa active agent and a carbidopa active agent in suitable amounts so that the levodopa active agent and carbidopa active agent are present in therapeutically effective amounts in the pharmaceutical composition. The levodopa active agent and carbidopa active agent may be added to water to produce a slurry. The slurry may be added to a mixture of one or more suspending agents (e.g., Carbopol® 971P or Carbopol® 974P), a neutralizing agent, and water as described herein to form a suspension. A neutralizing agent (e.g., sodium hydroxide) may be added to bring the pH within the aforementioned range. The suspension may or may not undergo N₂ sparging to reduce the oxygen level. Particularly, the suspension may be subjected to N₂ sparging. Optionally, the suspension may be degassed to remove any entrapped nitrogen or air from the suspension. The suspension may then be loaded into oxygen impermeable containers as described herein. Optionally, an oxygen scavenger may be added to the suspension as well. The combination of N₂ sparging of the suspension and use of lower oxygen permeability containers advantageously can result in a pharmaceutical composition with increased chemical stability by reducing both the initial solubilized O₂ present in the composition and the amount of O₂ ingress into the composition during storage.

In various aspects, the levodopa active agent can have any suitable particle size for preparing a pharmaceutical composition with the appropriate pharmacokinetic profile and maintaining the desired physical and chemical stability of the composition. Although a wide range of particle sizes can be used, the levodopa active agent (e.g., prior to forming the suspension) may have a particle size distribution where:
(i) D50 may be less than or equal to about 5 μm, less than or equal to about 3 μm, or less than or equal to about 1 μm;
(ii) D90 may be less than or equal to about 11 μm, less than or equal to about 9 μm, less than or equal to about 7 μm, less than or equal to about 5 μm or less than or equal to about 3 μm; and
(iii) D100 may be less than or equal to about 22 μm, less than or equal to about 21 μm, less than or equal to about 19 μm, less than or equal to about 17 μm, less than or equal to about 15 μm, less than or equal to about 13 μm or less than or equal to about 11 μm.

In one embodiment, the levodopa active agent has a particle size distribution of: (i) D50 less than or equal to about 5 μm; (ii) D90 less than or equal to 11 μm; and (iii) D100 less than or equal to 22 μm.

In another embodiment, the levodopa active agent (e.g., prior to forming the suspension) has a particle size distribution where:
(i) D10 is less than or equal to about 50 μm, less than or equal to about 20 μm, or less than or equal to about 10 μm;
(ii) D50 is less than or equal to about 100 μm, less than or equal to about 50 μm, or less than or equal to about 37 μm; and
(iii) D90 may be less than or equal to about 200 μm, less than or equal to about 150 μm, or less than or equal to about 120 μm.

In another embodiment, the levodopa active agent has a particle size distribution of: (i) D50 less than or equal to about 37.2 μm; (ii) D90 less than or equal to 121.4 μm; and (iii) D10 less than or equal to 9.8 μm.

The carbidopa active agent also can have any suitable particle size for preparing a pharmaceutical composition of the present disclosure with the appropriate pharmacokinetic profile and maintaining the desired physical and chemical stability of the composition. The carbidopa active agent (e.g., prior to forming the suspension) may have a particle size distribution where:
(i) D50 may be less than or equal to 3 μm;
(ii) D90 may be less than or equal to 7 μm, or less than or equal to 5 μm; and
(iii) D100 may be less than or equal to 21 μm, less than or equal to 19 μm, less than or equal to 17 μm, less than or equal to 15 μm, less than or equal to 13 μm, less than or equal to 11 μm, less than or equal to 9 μm.

In one embodiment, the carbidopa active agent, e.g., carbidopa monohydrate, may have a particle size distribution of: (i) D50 less than or equal to about 3 μm; (ii) D90 less than or equal to 7 μm; and (iii) D100 less than or equal to 21 μm.

In another embodiment, the carbidopa active agent has a particle size distribution of: (i) D50 less than or equal to about 9.4 μm; (ii) D90 less than or equal to 34.5 μm; and (iii) D10 less than or equal to 3.1 μm.

The levodopa active agent and/or the carbidopa active may be milled or micronized to achieve such a particle size distribution.

In another embodiment, the carbidopa active agent (e.g., prior to forming the suspension) may have a particle size distribution where:
(i) D10 is less than or equal to about 10 μm, less than or equal to about 5 μm, or less than or equal to about 3 μm;
(ii) D50 is less than or equal to about 20 μm, less than or equal to about 15 μm, or less than or equal to about 10 μm; and
(iii) D90 may be less than or equal to about 50 μm, less than or equal to about 40 μm, or less than or equal to about 35 μm.

In one embodiment, pharmaceutical compositions of the present disclosure comprising about 4 weight/weight percent of the levodopa active agent and about 1 weight/weight percent of the carbidopa active agent, e.g., carbidopa monohydrate, with the above described particle size distributions maintain physical stability for at least about 8 weeks, at least about 10 weeks, at least about 15 weeks, at least about 26 weeks, or at least about 60 weeks, so long as the yield value of the composition is at least about 0.3 Pa when measured after exposing the pharmaceutical composition to a temperature of about 5° C.

In another embodiment, pharmaceutical compositions of the present disclosure comprising about 4 weight/weight percent of the levodopa active agent and about 1 weight/weight percent of the carbidopa active agent, e.g., carbidopa monohydrate, with the above described particle size distributions maintain physical stability for at least about 8 weeks, at least about 10 weeks, at least about 15 weeks, at least about 26 weeks, or at least about 60 weeks, so long as the yield value of the composition is at least 0.34 Pa, at least about 0.4 Pa, at least about 0.5 Pa, at least about 0.6 Pa, at least about 0.7 Pa, at least about 0.8 Pa, at least about 0.9 Pa, at least about 1.0 Pa, at least about 1.1 Pa, at least about 1.2 Pa, at least about 1.3 Pa, at least about 1.4 Pa, at least about 1.5 Pa, at least about 1.6 Pa, at least about 1.7 Pa, at least about 1.8 Pa, at least about 1.9 Pa, at least about 2.0 Pa, at least about 2.1 Pa, at least about 2.2 Pa, at least about 2.3 Pa, at least about 2.4 Pa, at least about 2.5 Pa, at least about 2.6 Pa, at least about 2.7 Pa, at least about 2.8 Pa, at least about 2.9 Pa, or at least about 3.0 Pa, when measured after exposing the pharmaceutical composition to a temperature of about 5° C.

In another embodiment, pharmaceutical compositions comprising about 4 weight/weight percent of the levodopa active agent and about 1 weight/weight percent of the carbidopa active agent, e.g., carbidopa monohydrate, with the above described particle size distributions maintain physical stability for at least about 8 weeks, at least about 10 weeks, at least about 15 weeks, at least about 26 weeks, or at least about 60 weeks, so long as the yield value of the composition is at least about 0.3 Pa when measured after exposing the pharmaceutical composition to a temperature of about 25° C. and a relative humidity (RH) of about 60%.

In another embodiment, pharmaceutical compositions comprising about 4 weight/weight percent of the levodopa active agent and about 1 weight/weight percent of the carbidopa active agent, e.g., carbidopa monohydrate, with the above described particle size distributions maintain physical stability for at least about 8 weeks, at least about 10 weeks, at least about 15 weeks, at least about 26 weeks, or at least about 60 weeks, so long as the yield value of the composition is at least 0.34 Pa, at least about 0.4 Pa, at least about 0.5 Pa, at least about 0.6 Pa, at least about 0.7 Pa, at least about 0.8 Pa, at least about 0.9 Pa, at least about 1.0 Pa, at least about 1.1 Pa, at least about 1.2 Pa, at least about 1.3 Pa, at least about 1.4 Pa, at least about 1.5 Pa, at least about 1.6 Pa, at least about 1.7 Pa, at least about 1.8 Pa, at least about 1.9 Pa, at least about 2.0 Pa, at least about 2.1 Pa, at least about 2.2 Pa, at least about 2.3 Pa, at least about 2.4 Pa, at least about 2.5 Pa, at least about 2.6 Pa, at least about 2.7 Pa, at least about 2.8 Pa, at least about 2.9 Pa, or at least about 3.0 Pa, when measured after exposing the pharmaceutical composition to a temperature of about 25° C. and a RH of about 60%.

In another embodiment, pharmaceutical compositions comprising about 4 weight/weight percent of the levodopa active agent and about 1 weight/weight percent of the carbidopa active agent, e.g., carbidopa monohydrate, with the above described particle size distributions maintain physical stability for at least about 8 weeks, at least about 10 weeks, at least about 15 weeks, at least about 26 weeks, or at least about 60 weeks, so long as the yield value of the composition is at least about 0.3 Pa when measured after exposing the pharmaceutical composition to a temperature of about 40° C. and a RH of about 75%.

In another embodiment, pharmaceutical compositions comprising about 4 weight/weight percent of the levodopa active agent and about 1 weight/weight percent of the carbidopa active agent, e.g., carbidopa monohydrate, with the above described particle size distributions maintain physical stability for at least about 8 weeks, at least about 10 weeks, at least about 15 weeks, at least about 26 weeks, or at least about 60 weeks, so long as the yield value of the composition is at least 0.34 Pa, at least about 0.4 Pa, at least about 0.5 Pa, at least about 0.6 Pa, at least about 0.7 Pa, at least about 0.8 Pa, at least about 0.9 Pa, at least about 1.0 Pa, at least about 1.1 Pa, at least about 1.2 Pa, at least about 1.3 Pa, at least about 1.4 Pa, at least about 1.5 Pa, at least about 1.6 Pa, at least about 1.7 Pa, at least about 1.8 Pa, at least about 1.9 Pa, at least about 2.0 Pa, at least about 2.1 Pa, at least about 2.2 Pa, at least about 2.3 Pa, at least about 2.4 Pa, at least about 2.5 Pa, at least about 2.6 Pa, at least about 2.7 Pa, at least about 2.8 Pa, at least about 2.9 Pa, or at least about 3.0 Pa, when measured after exposing the pharmaceutical composition to a temperature of about 40° C. and a RH of about 75%.

The physical stability of a pharmaceutical composition of the present disclosure may be determined by its Acceptance Value (AV). A pharmaceutical composition is physically stable if the Acceptance Value (AV), defined by Equation 2, is no more than 15 for levodopa and no more than 15 for carbidopa.

AV=|M−X̅|+ks Equation 2.

The definition of each variable in Equation 2 is shown in Table 10.

TABLE 10
Definition of Variables Used in Calculating the Acceptance Value

| Variable | Definition | Conditions | Value |
|---|---|---|---|
| X̅ | Mean of individual contents (X₁, | | |
| | X₂, . . . , Xₙ), expressed as a | | |
| | percentage of the label claim | | |
| X₁, X₂, . . . , Xₙ | Individual contents of the units | | |
| | tested, expressed as a percentage | | |
| | of the label claim | | |
| n | Sample size (# of units in sample) | | |
| k | Acceptability constant | If n = 10, | then k = 2.4 |
| | | If n = 30, | then k = 2.0 |
| s | Sample standard deviation | | ${[ \frac{{\sum }_{i = 1}^{n} {( \text{?} - \overline{X} )}^{2}}{n - 1} ]}^{\frac{1}{2}}$ |
| RSD | Relative standard deviation (the | | 100s/X̅ |
| | sample standard deviation | | |
| | expressed as a percentage of | | |
| | the mean) | | |
| M (case 1) | Reference value to be applied | If 98.5% ≤ X̅ ≤ 101.5% | M = X̅ (AV = ks) |
| | where T ≤ 101.5 | If X̅ < 98.5% | M = 98.5% |
| | | | (AV = 98.5 − X̅ + ks) |
| | | If X̅ >101.5% | M = 101.5% |
| | | | (AV = X̅ − 101.5 + ks) |
| M (case 2) | Reference value to be applied | If 98.5 ≤ X̅ ≤ T | M = X̅ (AV = ks) |
| | where T > 101.5 | If X̅ < 98.5% | M = 98.5% |
| | | | (AV = 98.5 − X̅ + ks) |
| | | If X̅ > T | M = T% |
| | | | (AV = X̅ − T + ks) |

$\text{?} \text{indicates text missing or illegible when filed}$

In some embodiments, the AV for the levodopa active agent in the pharmaceutical composition is about 15. In another embodiment, the AV for the levodopa active agent in the pharmaceutical composition is less than about 15, e.g., about 14, about 13, about 12, about 11, about 10, about 9, about 8, about 7, about 6, about 5, about 4, about 3, about 2, or about 1. In some embodiments, the AV for the levodopa active agent is measured after storing the pharmaceutical composition in a foil pouch for at least about 8 weeks, at least about 10 weeks, at least about 15 weeks, at least about 26 weeks, or at least about 60 weeks.

In some embodiments, the AV for the carbidopa active agent in the pharmaceutical composition is about 15. In another embodiment, the AV for the levodopa active agent in the pharmaceutical composition is less than about 15, e.g., about 14, about 13, about 12, about 11, about 10, about 9, about 8, about 7, about 6, about 5, about 4, about 3, about 2, or about 1. In some embodiments, the AV for the carbidopa active agent is measured after storing the pharmaceutical composition in a foil pouch for at least about 8 weeks, at least about 10 weeks, at least about 15 weeks, at least about 26 weeks, or at least about 60 weeks.

In another embodiment, the levodopa active agent and the carbidopa active agent with the above described particle size distributions may successfully form a suspension and maintain physical stability of the suspension throughout the pharmaceutical composition's shelf life even when the levodopa active agent and the carbidopa active agent are present at higher concentrations in the composition as long as the yield value of the composition is at least 1.1 Pa. For example, physical stability may be maintained even when the pharmaceutical composition comprises about 4 weight/weight percent of levodopa active agent and 1 weight/weight percent carbidopa active agent (e.g., carbidopa monohydrate). In another embodiment, the yield of the composition is at least about 0.3 Pa.

In another embodiment, the yield value of the pharmaceutical composition comprising about 4 weight/weight percent of levodopa active agent and 1 weight/weight percent carbidopa active agent is at least about 2.0 Pa. In another embodiment, the yield value is at least about 3.0 Pa. In another embodiment, the yield value is at least about 4.0 Pa. In another embodiment, the yield value is at least about 5.0 Pa. In another embodiment, the yield value is at least about 6.0 Pa. In another embodiment, the yield value is at least about 7.0 Pa. In another embodiment, the yield value is at least about 8.0 Pa. In another embodiment, the yield value is at least about 9.0 Pa. In another embodiment, the yield value is or at least about 10.0 Pa.

In another embodiment, pharmaceutical compositions as described herein prepared by the methods described herein are provided. In particular, a levodopa active agent and a carbidopa active agent may be provided in suitable amounts so that the levodopa active agent and carbidopa active agent are present in therapeutically effective amounts in the pharmaceutical composition. The levodopa active agent and the carbidopa active agent provided have a particle size distribution as described above. The levodopa active agent and carbidopa active agent are added to water to produce a slurry. The slurry is added to a mixture that may contain one or more suspending agents, (e.g., Carbopol® 971P or Carbopol® 974P), a neutralizing agent, and water as described herein The suspension can undergo N₂ sparging to reduce the oxygen level. The suspension can be loaded into lower oxygen permeability or oxygen impermeable containers as described herein. Optionally, an oxygen scavenger may be added to the suspension as well.

### IV. Methods of Treatment

The present disclosure further provides methods of treating Parkinson's disease and associated conditions comprising administering a therapeutically effective amount of a pharmaceutical composition comprising a high concentration levodopa active agent and carbidopa active agent to a patient. A pharmaceutical composition comprising a high concentration levodopa active agent and carbidopa active agent can comprise, for example, a liquid or viscous liquid comprising about 200 mg levodopa and about 50 mg carbidopa monohydrate per each 5.0 mL volume.

In one embodiment, the present disclosure provides a method of treating a condition in need of treatment, wherein the method comprises administering to the patient a therapeutically effective amount of a pharmaceutical composition of the present disclosure.

In one embodiment, the condition treated by administering the pharmaceutical composition is Parkinson's disease.

In another embodiment, the condition treated by administering the pharmaceutical composition is impaired motor performance in a patient with Parkinson's disease (i.e., a method of improving motor performance in a patient with Parkinson's disease).

In another embodiment, the pharmaceutical composition is administered to treat motor fluctuations in a patient with Parkinson's disease.

In another embodiment, the pharmaceutical composition is administered to treat dyskinesia in a patient with Parkinson's disease.

In another embodiment, the present pharmaceutical compositions are administered via intestinal administration. They can be administered (or “infused”) directly into the intestine, such as the small intestine (e.g., duodenum or the jejunum) by a permanent tube inserted via percutaneous endoscopic gastrostomy, for example, with an outer transabdominal tube and an inner intestinal tube. In one aspect, the first compound and the second compound are administered via a tube inserted by radiological gastrojejunostomy. In another aspect, the present pharmaceutical compositions are administered via a temporary nasoduodenal tube that is inserted into the patient, for example to initially to determine if the patient responds favorably to the treatment method before the permanent tube is inserted.

In embodiments where one or more of the present pharmaceutical compositions are administered via intestinal administration, administration can be carried out using a portable pump, such as the pump sold under the trade name, CADD-Legacy Duodopa® pump. Specifically, a cassette, pouch, vial or cartridge comprising the first compound and the second compound can be attached to the pump to create the delivery system. The delivery system is then connected to the nasoduodenal tube, the transabdominal port, the duodenal tube, or the jejunum tube for intestinal administration.

In one embodiment, the method comprises administering one or more of the present pharmaceutical compositions to the patient substantially continuously over a period of at least about 12 hours. In additional aspects, the present pharmaceutical compositions can be administered substantially continuously over a period of about 16 hours, about 24 hours, about 36 hours, about 48 hours, about 3 days, about 4 days, about 5 days, about 6 days, about one week, or longer.

In one embodiment, the dosing of the present pharmaceutical composition administered to the patient is adjusted to optimize the clinical response achieved by a patient, which means, for example, maximizing the functional ON-time during the day by minimizing the number and duration of OFF-time episodes (i.e., bradykinesia) and minimizing ON-time with disabling dyskinesia.

In one embodiment, the daily dose of levodopa active agent administered to the patient according to methods of the present disclosure may be, for example, about 20 to about 5000 mg, about 20 mg to about 4000 mg, about 20 mg to about 3000 mg, about 20 mg to about 2000 mg, or about 20 mg to about 1000 mg per day. In various aspects, the patient may receive, for example, about: 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg of levodopa active agent per day.

In one embodiment, the daily dose of the carbidopa active agent administered to the patient according to methods of the present disclosure may be, for example, 0 to about 625 mg, 0 mg to about 500 mg, 0 mg to about 375 mg, 0 mg to about 250 mg, or 0 mg to about 125 mg per day. In various aspects, the patient may receive, for example, about: 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, or 1250 mg of carbidopa active agent per day.

In some embodiments, an amount of levodopa active agent and carbidopa active agent are administered such that in combination they are sufficient to achieve an L-dopa plasma level in the patient of at least about 100 ng/mL. In one aspect, the L-dopa plasma level is at least about 200 ng/mL. In another aspect, the L-dopa plasma level is at least about 200 ng/mL. In another aspect, the L-dopa plasma level is at least about 300 ng/mL. In another aspect, the L-dopa plasma level is at least about 400 ng/mL. In another aspect, the L-dopa plasma level is at least about 500 ng/mL. In another aspect, the L-dopa plasma level is at least about 600 ng/mL. In another aspect, the L-dopa plasma level is at least about 700 ng/mL. In another aspect, the L-dopa plasma level is at least about 800 ng/mL. In another aspect, the L-dopa plasma level is at least about 900 ng/mL. In another aspect, the L-dopa plasma level is at least about 1,000 ng/mL. In another aspect, the L-dopa plasma level is at least about 1,500 ng/mL. In another aspect, the L-dopa plasma level is at least about 2,000 ng/mL. In another aspect, the L-dopa plasma level is at least about 3,000 ng/mL. In another aspect, the L-dopa plasma level is at least about 4,000 ng/mL. In another aspect, the L-dopa plasma level is at least about 5,000 ng/mL.

In some embodiments, an amount of the levodopa active agent and carbidopa active agent are administered such that in combination they are sufficient to achieve an L-dopa plasma level from about 10 ng/mL to about 8,000 ng/mL. In one aspect, the L-dopa plasma level is from about 25 ng/mL to about 6,000 ng/mL. In another aspect, the L-dopa plasma level is from about 50 ng/mL to about 4,000 ng/mL. In another aspect, the L-dopa plasma level is from about 100 ng/mL to about 2,000 ng/mL. In another aspect, the L-dopa plasma level is from about 25 ng/mL to about 1,200 ng/mL. In another aspect, the L-dopa plasma level is from about 10 ng/mL to about 500 ng/mL. In another aspect, the L-dopa plasma level is from about 25 ng/mL to about 500 ng/mL.

In some embodiments, the above-described L-dopa concentration ranges are maintained for at least about: a 1 hour interval, a 2 hour interval, a 3 hour interval, a 4 hour interval, a 5 hour interval, a 6 hour interval, a 7 hour interval, an 8 hour interval, a 9 hour interval, a 10 hour interval, an 11 hour interval, a 12 hour interval, an 18 hour interval, or a 24 hour interval.

In some embodiments, an amount of the levodopa active agent and carbidopa active agent are administered such that in combination they are sufficient to maintain a carbidopa plasma level less than about 500 ng/mL. In one aspect, the carbidopa plasma level is less than about 250 ng/mL. In another aspect, the carbidopa plasma level is less than about 100 ng/mL. In another aspect, the carbidopa plasma level is less than about 50 ng/mL. In another aspect, the carbidopa plasma level is less than about 25 ng/mL.

In some embodiments, an amount of the levodopa active agent and carbidopa active agent are administered such that in combination they are sufficient to maintain a carbidopa plasma level from about 1 to about 10 ng/mL. In one aspect, the carbidopa plasma level is from about 1 to about 25 ng/mL. In another aspect, the carbidopa plasma level is from about 1 to about 50 ng/mL. In another aspect, the carbidopa plasma level is from about 1 to about 100 ng/mL. In another aspect, the carbidopa plasma level is from about 1 to about 250 ng/mL. In another aspect, the carbidopa plasma level is from about 5 to about 250 ng/mL. In another aspect, the carbidopa plasma level is from about 5 to about 100 ng/mL. In another aspect, the carbidopa plasma level is from about 10 to about 250 ng/mL. In another aspect, the carbidopa plasma level is from about 10 to about 100 ng/mL. In another aspect, the carbidopa plasma level is from about 25 to about 250 ng/mL. In another aspect, the carbidopa plasma level is from about 25 to about 100 ng/mL.

In some embodiments, the above-described carbidopa concentration ranges are maintained for at least about: a 1 hour interval, a 2 hour interval, a 3 hour interval, a 4 hour interval, a 5 hour interval, a 6 hour interval, a 7 hour interval, an 8 hour interval, a 9 hour interval, a 10 hour interval, an 11 hour interval, a 12 hour interval, an 18 hour interval, or a 24 hour interval.

In additional embodiments, the levodopa active agent and the carbidopa active agent administered may have a particle size distribution of as described above.

In various embodiments, the pharmaceutical composition may be present in a container as described above and prior to administration to the patient, the gel-suspension may or may not be subjected to N₂ sparging. When the gel-suspension is subjected to N₂ sparging and the container has low oxygen permeability, the pharmaceutical composition may not experience degradation producing DHPA at a rate faster than 0.04 w/w % per week of refrigerated storage. (The percent is relative to the label amount of carbidopa.) Additionally or alternatively, when the container is subjected to N₂ sparging, the pharmaceutical composition may not experience degradation producing DHPPA at a rate faster than 0.04 w/w % per week of refrigerated storage. (The percent is relative to the label amount of carbidopa.) Additionally or alternatively, when the container is subjected to N₂ sparging, the pharmaceutical composition may not degrade producing hydrazine at a rate faster than 0.6 μg/g per week of refrigerated storage, where μg/g denotes μg of hydrazine per gram of gel-suspension.

### V. Co-Administration of Additional Therapeutic Agents

The methods of treatment of the present disclosure optionally can further comprise administration of one or more therapeutic agents for the treatment of Parkinson's disease in addition to administration of the levodopa active agent and carbidopa active agent. In one embodiment, the additional therapeutic agent(s) is selected from the group consisting of decarboxylase inhibitors other than a carbidopa active agent (e.g., benserazide), catechol methyl transferase (“COMT”) inhibitors (e.g., entacapone and tolcapone), and monoamine oxidase A (“MAO-A”) or monoamine oxidase B (“MAO-B”) inhibitors (e.g., moclobemide, rasagiline, selegiline, and safinamide). In one aspect, the additional therapeutic agent(s) is selected from the group consisting of decarboxylase inhibitors other than a carbidopa active agent. In another aspect, the additional therapeutic agent(s) is selected from the group consisting of COMT inhibitors. In another aspect, the additional therapeutic agent(s) is selected from the group consisting of MAO-A inhibitors. In another aspect, the additional therapeutic agent(s) is selected from the group consisting of MAO-B inhibitors.

In a similar manner, the pharmaceutical compositions of the present disclosure optionally can further comprise one or more additional therapeutic agents for the treatment of Parkinson's disease as described above.

### VI. Kits

The present disclosure also provides kits comprising one or more pharmaceutical dosage forms comprising a carbidopa active agent; kits comprising one or more pharmaceutical dosage forms comprising a levodopa active agent; and kits comprising one or more pharmaceutical dosage forms comprising both a levodopa active agent and carbidopa active agent. In the kit, the pharmaceutical dosage forms may be present, separately or together, in a lower O₂ permeability bag. The pharmaceutical dosage forms may comprise a high concentration of a levodopa active agent and a carbidopa active agent, for example, a levodopa active agent in an amount of about 4.0 weight/weight percent of the total composition; and a carbidopa monohydrate active agent in an amount of about 1.0 weight/weight percent of the total composition. The kit optionally can comprise one or more additional therapeutic agents and/or instructions, for example, instructions for using the kit to treat a patient having Parkinson's disease or an associated condition.

### VII. Examples

The following non-limiting examples are provided to further illustrate the present disclosure. Abbreviations used in the examples below include the following:

“Cmax” means maximum observed plasma concentration.

“Tmax” means time to maximum observed plasma concentration.

“AUC” means area under the plasma concentration-time curve.

“t_{1/2}” means biological half-life, i.e., the time required for half the quantity of a drug or other substance administered to a living organism to be metabolized or eliminated by normal biological processes.

### VIII. Particular Embodiments

The disclosure provides Embodiments I-XX1 as particular embodiments.

Embodiment I. A pharmaceutical composition comprising a levodopa active agent and a carbidopa active agent for intraduodenal administration wherein the levodopa active agent and the carbidopa active agent are suspended in one or more polymer-based suspending agents, and wherein the pharmaceutical composition has a yield value of at least about 1.1 Pa.

Embodiment II. The pharmaceutical composition according to Embodiment I, wherein the pharmaceutical composition comprises:

a levodopa active agent in an amount of about 4.0 weight/weight percent of the total composition;

a carbidopa monohydrate active agent in an amount of about 1.0 weight/weight percent of the total composition; and.

a liquid vehicle.

Embodiment III. The pharmaceutical composition according to Embodiments I or II, wherein a polymer-based suspending agent is an acrylic acid-based polymer.

Embodiment IV. The pharmaceutical composition according to Embodiments I or II, wherein a polymer-based suspending agent is a hydrocolloid polymer.

Embodiment V. The pharmaceutical composition according to Embodiment III, wherein a polymer-based suspending agent is a carbomer.

Embodiment VI. The pharmaceutical composition according to Embodiment III, wherein a polymer-based suspending agent is a carboxypolymethylene and carbomer polymer sold under the trade name Carbopol® 971P or Carbopol® 974P.

Embodiment VII. The pharmaceutical composition according to Embodiment IV, wherein a polymer-based suspending agent is selected from the group consisting of locust beam gum, guar gum, methylcellulose, sodium carboxymethylcellulose with microcrystalline cellulose and gum tragacanth.

Embodiment VIII. The pharmaceutical composition according to Embodiments I-VII, wherein the concentration of the liquid vehicle is in an amount of from about zero percent to about 95 weight/weight percent of the total composition.

Embodiment IX. The pharmaceutical composition according to Embodiment VIII, wherein the liquid vehicle is selected from the group consisting of water or polyethylene glycol.

Embodiment X. The pharmaceutical composition according to Embodiment VIII, wherein the liquid vehicle is water only.

Embodiment XI. The pharmaceutical composition according to any one of Embodiments I-X, wherein the amount of impurities in the pharmaceutical composition is in an amount of less than 5.8 w/w % of the total weight of the composition when maintained at a temperature of about 20-25° C. for a period of at least 15 weeks.

Embodiment XII. The pharmaceutical composition according to any one of Embodiment I-XI, wherein the pharmaceutical composition is present in a primary or secondary container that acts as an oxygen barrier.

Embodiment XIII. A pharmaceutical dosage form comprising the pharmaceutical composition of any one of Embodiment I-XII in a disposable drug reservoir having an oxygen impermeable enclosure disposed therein, wherein the oxygen impermeable enclosure is purged with an inert gas and an oxygen scavenger is added.

Embodiment XIV. The pharmaceutical dosage form according to Embodiment XIII, wherein the pharmaceutical dosage form is suitable for use in a continuous infusion pump capable of delivering the composition in a therapeutically effective manner.

Embodiment XI. A method of preparing the pharmaceutical composition comprising a levodopa active agent and a carbidopa active agent for intraduodenal administration, wherein the method comprises:

adding one or more suspending agents to water to disperse the polymer;

adding a neutralizing agent to bring the pH about 6.5 to make the medium;

adding a levodopa active agent and a carbidopa active agent to water to form a slurry;

adding the slurry to the medium to form a suspension; and

optionally subjecting the suspension to N₂ sparging.

Embodiment XVI. The method according to Embodiment XV, further comprising loading the suspension into a lower oxygen permeability container.

Embodiment XVII. The method according to claim Embodiment XV, wherein a suspending agent is an acrylic acid-based polymer.

Embodiment XVIII. The method according to Embodiment XV, wherein the polymer-based suspending agent is Carbopol® 971P or Carbopol® 974P.

Embodiment XX. The method according to Embodiment XV, wherein the neutralizing agent is sodium hydroxide.

Embodiment XXI. A method of treating Parkinson's disease in a patient in need thereof, wherein the method comprises administering to the patient a pharmaceutical composition comprising a levodopa active agent and a carbidopa active agent for intraduodenal administration, wherein the levodopa active agent and carbidopa active agent are provided in a therapeutically effective manner for the patient and suspended in one or more polymer-based suspending agents, and wherein the pharmaceutical composition has a yield value of at least about 1.1 Pa.

The disclosure also provides Embodiments 1-50 as particular embodiments.

Embodiment 1. A pharmaceutical composition for intraduodenal administration comprising:

(a) a levodopa active agent in an amount of about 4.0 w/w % of the total composition;

(b) a carbidopa active agent in an amount of about 1.0 w/w % of the total composition;

(c) a polymer-based suspending agent in an amount of about 0.1 w/w % to about 5 w/w % of the total composition; and

(d) a liquid vehicle,

wherein:

(i) the liquid vehicle is water, polyethylene glycol, or a mixture of water and polyethylene glycol;

(ii) the acceptance value of the pharmaceutical composition is less than or equal to 15 with respect to the levodopa active agent and less than or equal to 15 with respect to the carbidopa active agent; and

(iii) the yield value of the pharmaceutical composition is at least about 0.3 Pa,

wherein:

the acceptance value and yield value are measured after exposing the pharmaceutical composition to a temperature of about 25° C. and relative humidity of about 60% for a period of at least about 8 weeks.

According to Embodiment 1, “a polymer-based suspending agent” may represent one or more polymer-based suspending agents. When two polymer-based suspending agents are included in the pharmaceutical composition, both may be acrylic acid-based polymers, e.g., Carbopol® 971P and Carbopol® 974P, both may be hydrocolloid polymers, e.g., Avicel® CL-611 and Avicel® RC 591, or one may be an acrylic acid-based polymer and the other may be a hydrocolloid polymer, e.g., Carbopol® 971P and Avicel® CL-61.

Embodiment 2. The pharmaceutical composition according to Embodiment 1, wherein acceptance value and yield value are measured after exposing the pharmaceutical composition to a temperature of about 25° C. and relative humidity of about 60% for a period of at least about 15 weeks.

Embodiment 3. The pharmaceutical composition according to Embodiment 1, wherein acceptance value and yield value are measured after exposing the pharmaceutical composition to a temperature of about 25° C. and relative humidity of about 60% for a period of at least about 26 weeks.

Embodiment 4. The pharmaceutical composition according to Embodiment 1, wherein acceptance value and yield value are measured after exposing the pharmaceutical composition to a temperature of about 25° C. and relative humidity of about 60% for a period of at least about 60 weeks.

Embodiment 5. The pharmaceutical composition according to any one of Embodiments 1-4, wherein the polymer-based suspending agent is an acrylic acid-based polymer.

Embodiment 6. The pharmaceutical composition according to any one of Embodiments 1-5, wherein the polymer-based suspending agent is a carbomer, e.g., Carbopol® 934P, Carbopol® 971P, or Carbopol® 974P.

Embodiment 7. The pharmaceutical composition according to any one of Embodiments 1-6, wherein the polymer-based suspending agent is Carbopol® 971P or Carbopol® 974P, or a combination thereof.

Embodiment 8. The pharmaceutical composition according to any one of Embodiments 1-7, wherein the composition comprises the polymer-based suspending agent in an amount of about 0.1 w/w % to about 0.3 w/w % of the total composition, e.g., about 0.11 w/w %, about 0.12 w/w %, about 0.13 w/w %, about 0.13 w/w %, about 0.14 w/w %, about 0.15 w/w %, about 0.16 w/w %, about 0.17 w/w %, about 0.18 w/w %, about 0.19 w/w %, about 0.20 w/w %, about 0.21 w/w %, about 0.22 w/w %, about 0.23 w/w %, about 0.24 w/w %, about 0.25 w/w %, about 0.26 w/w %, about 0.27 w/w %, about 0.28 w/w %, or about 0.29 w/w % of the total composition.

Embodiment 9. The pharmaceutical composition according to any one of Embodiments 1-4, wherein the polymer based suspending agent is a hydrocolloid polymer.

Embodiment 10. The pharmaceutical composition according to Embodiments 1 or 9, wherein the polymer-based suspending agent is selected from the group consisting of locust beam gum, guar gum, methylcellulose, sodium carboxymethylcellulose with microcrystalline cellulose, xanthan gum, and gum tragacanth, or a combination thereof.

Embodiment 11. The pharmaceutical composition according to any one of Embodiments 1, 9, or 10, wherein the polymer-based suspending agent is selected from the group consisting of locust beam gum, guar gum, xanthan gum, and gum tragacanth, or a combination thereof.

Embodiment 12. The pharmaceutical composition according to any one of Embodiments 1, 9, or 10, wherein the polymer-based suspending agent is selected from the group consisting methylcellulose and sodium carboxymethylcellulose with microcrystalline cellulose, or a combination thereof.

Embodiment 13. The pharmaceutical composition according to any one of Embodiments 1, 9, or 10, wherein the polymer-based suspending agent is sodium carboxymethylcellulose with microcrystalline cellulose, e.g., Avicel® CL-611 or Avicel® RC 591.

Embodiment 14. The pharmaceutical composition according to any one of Embodiments 1 or 9-13, wherein the composition comprises the polymer-based suspending agent in an amount of about 1 w/w % to about 5 w/w % of the total composition.

Embodiment 15. The pharmaceutical composition according to any one of Embodiments 1-14, wherein the liquid vehicle is a mixture of water and polyethylene glycol.

Embodiment 16. The pharmaceutical composition according to any one of Embodiments 1-14, wherein the liquid vehicle is water.

Embodiment 17. The pharmaceutical composition according to any one of Embodiments 1-5 comprising:

(a) levodopa in an amount of about 4.0 w/w % of the total composition;

(b) carbidopa monohydrate in an amount of about 1.0 w/w % of the total composition;

(c) Carbopol® 971P or Carbopol® 974P in an amount of about 0.1 w/w % to about 0.2 w/w % of the total composition; and

(d) water.

Embodiment 18. The pharmaceutical composition according to Embodiment 17 consisting essentially of:

(a) levodopa in an amount of about 4.0 w/w % of the total composition;

(b) carbidopa monohydrate in an amount of about 1.0 w/w % of the total composition;

(c) Carbopol® 971P or Carbopol 974P in an amount of about 0.1 w/w % to about 0.2 w/w % of the total composition;

(d) a neutralizing agent; and

(e) water.

Embodiment 19. The pharmaceutical composition according to Embodiment 18 consisting of:

(a) levodopa in an amount of about 4.0 w/w % of the total composition;

(b) carbidopa monohydrate in an amount of about 1.0 w/w % of the total composition;

(c) Carbopol® 971P or Carbopol® 974P in an amount of about 0.1 w/w % to about 0.2 w/w % of the total composition;

(d) sodium hydroxide in an amount of about 0.01 w/w % to about 0.3 w/w %; and

(e) water.

Embodiment 20. The pharmaceutical composition according to any one of Embodiments 1-19, wherein the pharmaceutical composition has a yield value of at least about 0.34 Pa.

Embodiment 21. The pharmaceutical composition according to Embodiment 20, wherein the pharmaceutical composition has a yield value of at least about 0.6 Pa.

Embodiment 22. The pharmaceutical composition according to Embodiment 21, wherein the pharmaceutical composition has a yield value of at least about 0.9 Pa.

Embodiment 23. The pharmaceutical composition according to Embodiment 22, wherein the pharmaceutical composition has a yield value of at least about 1.1 Pa.

Embodiment 24. The pharmaceutical composition according to any one of Embodiments 1-18 or 20-23 further comprising a neutralizing agent in an amount of about 0.01 w/w % to about 0.5 w/w % of the total composition.

Embodiment 25. The pharmaceutical composition according to Embodiment 24, wherein the neutralizing agent is sodium hydroxide.

Embodiment 26. The pharmaceutical composition according to any one of Embodiments 1-25, wherein the levodopa active agent has a median particle size distribution (D50) of ≤100 μm.

Embodiment 27. The pharmaceutical composition according to Embodiment 26, wherein the levodopa active agent has a median particle size distribution (D50) of ≤37 μm.

Embodiment 28. The pharmaceutical composition according to Embodiment 27, wherein the levodopa active agent has a median particle size distribution (D50) of ≤5 μm.

Embodiment 29. The pharmaceutical composition according to any one of Embodiments 1-28, wherein the carbidopa active agent has a median particle size distribution (D50) of ≤20 μm.

Embodiment 30. The pharmaceutical composition according to Embodiment 29, wherein the carbidopa active agent has a median particle size distribution (D50) of about ≤10 μm.

Embodiment 31. The pharmaceutical composition according to Embodiment 30, wherein the carbidopa active agent has a median particle size distribution (D50) of ≤3 μm.

Embodiment 32. The pharmaceutical composition according to any one of Embodiments 1-31, wherein the amount of impurities in the pharmaceutical composition is in an amount of less than about 5.8 w/w %, e.g., less than about 5 w/w %, less than about 4 w/w %, less than about 3 w/w %, less than about 2 w/w %, or less than about 1 w/w %, of the total weight of the composition when maintained at a temperature of about 20-25° C. and a relative humidity of about 60% for a period of at least 8 weeks.

Embodiment 33. The pharmaceutical composition according to any one of Embodiments 1-31, wherein the amount of impurities in the pharmaceutical composition is in an amount of less than about 5.8 w/w %, e.g., less than about 5 w/w %, less than about 4 w/w %, less than about 3 w/w %, less than about 2 w/w %, or less than about 1 w/w %, of the total weight of the composition when maintained at a temperature of about 20-25° C. and a relative humidity of about 60% for a period of at least 15 weeks.

Embodiment 34. The pharmaceutical composition according to any one of Embodiments 1-31, wherein the amount of impurities in the pharmaceutical composition is in an amount of less than about 5.8 w/w %, e.g., less than about 5 w/w %, less than about 4 w/w %, less than about 3 w/w %, less than about 2 w/w %, or less than about 1 w/w %, of the total weight of the composition when maintained at a temperature of about 20-25° C. and a relative humidity of about 60% for a period of at least 26 weeks.

Embodiment 35. The pharmaceutical composition according to any one of Embodiments 1-31, wherein the amount of impurities in the pharmaceutical composition is in an amount of less than about 5.8 w/w %, e.g., less than about 5 w/w %, less than about 4 w/w %, less than about 3 w/w %, less than about 2 w/w %, or less than about 1 w/w %, of the total weight of the composition when maintained at a temperature of about 20-25° C. and a relative humidity of about 60% for a period of at least 60 weeks.

Embodiment 36. The pharmaceutical composition according to any one of Embodiments 1-35, wherein the pharmaceutical composition is present in a primary or secondary container that acts as an oxygen barrier, e.g., a foil pouch.

Embodiment 37. A pharmaceutical dosage form comprising the pharmaceutical composition according to any one of Embodiments 1-35 in a disposable drug reservoir having an oxygen impermeable enclosure disposed therein, wherein the oxygen impermeable enclosure is purged with an inert gas and an oxygen scavenger is added.

Embodiment 38. The pharmaceutical dosage form according to Embodiment 37, wherein the pharmaceutical dosage form is suitable for use in a continuous infusion pump capable of delivering the composition in a therapeutically effective manner.

Embodiment 39. A method of preparing the pharmaceutical composition comprising a levodopa active agent and a carbidopa active agent for intraduodenal administration, wherein

(i) the pharmaceutical composition has an acceptance value of the pharmaceutical composition is less than or equal to 15 with respect to the levodopa active agent and less than or equal to 15 with respect to the carbidopa active agent; and

(ii) the pharmaceutical composition has a yield value of at least about 0.3 Pa,

wherein:

the acceptance value and yield value are measured after exposing the pharmaceutical composition to a temperature of about 25° C. and relative humidity of about 60% for a period of at least about 8 weeks,

the method comprising:

adding a acrylic acid-based polymer suspending agent to water to form a dispersion;

adding a neutralizing agent to the dispersion to bring the pH to about 6.5 to form a medium;

adding a levodopa active agent and a carbidopa active agent to water to form a slurry; and

adding the slurry to the medium to form the pharmaceutical composition.

Embodiment 40. The method according to Embodiment 39, wherein the acrylic acid-based polymer suspending agent is a carbomer.

Embodiment 41. The pharmaceutical composition according to Embodiment 39, wherein the acrylic acid-based polymer suspending agent is Carbopol® 971P or Carbopol® 974P.

Embodiment 42. The method according to any one of Embodiments 38-40, wherein the neutralizing agent is sodium hydroxide.

Embodiment 43. A method of preparing the pharmaceutical composition comprising a levodopa active agent and a carbidopa active agent for intraduodenal administration, wherein

(i) the pharmaceutical composition has an acceptance value of the pharmaceutical composition is less than or equal to 15 with respect to the levodopa active agent and less than or equal to 15 with respect to the carbidopa active agent; and

(ii) the pharmaceutical composition has a yield value of at least about 0.3 Pa,

wherein:

the acceptance value and yield value are measured after exposing the pharmaceutical composition to a temperature of about 25° C. and relative humidity of about 60% for a period of at least about 8 weeks,

the method comprising:

adding a hydrocolloid polymer suspending agent to water to form a dispersion;

adding a levodopa active agent and a carbidopa active agent to water to form a slurry; and

adding the slurry to the medium to form the pharmaceutical composition.

Embodiment 44. The pharmaceutical composition according to Embodiment 43, wherein the polymer-based suspending agent is selected from the group consisting of locust beam gum, guar gum, sodium carboxymethylcellulose with microcrystalline cellulose, xanthan gum, and gum tragacanth.

Embodiment 45. The method according to any one of Embodiments 39-44 further comprising subjecting the pharmaceutical composition to N₂ sparging.

Embodiment 46. The method according to any one of Embodiments 39-45, further comprising loading the pharmaceutical composition in to a lower oxygen permeability container.

Embodiment 47. A method of treating Parkinson's disease in a patient in need thereof, the method comprising administering to the patient a therapeutically effective amount of the pharmaceutical composition according to any one of Embodiments 1-36.

Embodiment 48. The pharmaceutical composition according to any one of Embodiments 1-36, wherein the acceptance value for the levodopa active agent is less than or equal to 15, e.g., about 14, about 13, about 12, about 11, about 10, about 9, about 8, about 7, about 6, about 5, about 4, about 3, about 2, or about 1.

Embodiment 49. The pharmaceutical composition according to any one of Embodiments 1-36 or 48, wherein the acceptance value for the carbidopa active agent is less than or equal to 15, e.g., about 14, about 13, about 12, about 11, about 10, about 9, about 8, about 7, about 6, about 5, about 4, about 3, about 2, or about 1.

Embodiment 50. The pharmaceutical dosage form according to Embodiments 37 or 38 comprising about 40 mg/mL or the levodopa active agent and about 10 mg/mL of the carbidopa active agent.

### Example 1: Preparation of Pharmaceutical Composition

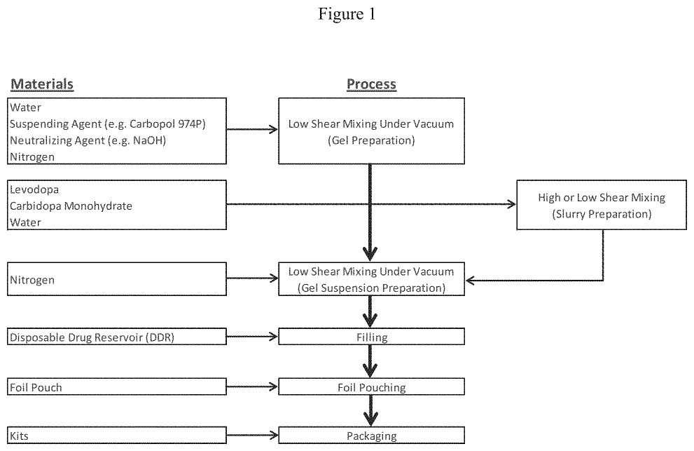

A levodopa active agent/carbidopa active agent pharmaceutical composition was prepared with Carbopol® 974P as shown in FIG. 1 and as described below:

### 1.1 Slurry Preparation

Levodopa and Carbidopa were added to water in a vessel and mixed using a high shear mixer. Alternatively the overhead impeller can be replaced with a low shear mixer. The slurry was used to wet and delump the API (drug actives).

### 1.2 Gel-Suspension Preparation

Carbopol 974P was added to water in a second vessel and was mixed under vacuum using a low shear mixer. Sodium hydroxide was added to mixture to neutralize the polymer. The slurry was added to the mixture and was further mixed using the low shear mixer to complete the gel-suspension preparation.

### 1.3 Filling and Pouching

The gel-suspension was filled into disposable drug reservoirs (DDRs). Fill weight was checked at routine intervals via a balance. The filled DDR was covered with a foil pouch. The pouch was sparged with nitrogen and was heat sealed.

### 1.4 Packaging

The foil pouches were packaged into a kit that holds 7 DDRs. The kits were then stored at various conditions—at 5° C., at 25° C. and about 60% relative humidity (RH), and at 40° C. and 75% RH.

TABLE 1
Formulation of Pharmaceutical Composition

| Component | w/w % |
|---|---|
| Levodopa micronized | 4.0 |
| Carbidopa monohydrate micronized | 1.0 |
| Carbopol ® 974P | 0.18 |
| Sodium hydroxide | 0.055 |
| Water | 94.76 |

TABLE 2
Drug Product and API Attributes

| Drug Product and API Attributes | Value |
|---|---|
| Yield Value (Pa) at 25° C. | ≥4 |
| Levodopa Particle Size (μm) | ≤22 |
| Carbidopa Monohydrate Particle Size (μm) | ≤21 |
| Oxygen Concentration | ≤Ambient |
| pH | 3-8 |

### Example 2: High Concentration Pharmaceutical Composition Stability

### 2.1—Formulation Preparation

A Carbopol 974P based formulation was made on pilot scale equipment. The overall manufacturing process was the same as in Example 1. The batch was filled into DDRs (Disposable Drug Reservoirs) The DDR samples were then packaged in nitrogen sparged foil pouches to prevent any oxygen ingression from the environment. The samples maintained their chemical and physical stability throughout the 15 week stability study at 25° C. This result cannot be achieved with other levodopa carbidopa intestinal gels (e.g. on market Duodopa).

TABLE 3
Drug Product and API Material Attributes for
Carbopol ® Based Formulation

| Drug Product and API Attributes | Value* | |
|---|---|---|
| Yield Value at 25° C. | 6.9 | Pa |
| Levodopa Particle Size | ≤22 | μm |
| Carbidopa Monohydrate Particle Size | ≤21 | μm |
| Oxygen Concentration | Ambient | |
| pH | 6.5 | |
| Batch Size | 9 | Kg |

*Values were measured in process

### 2.2—Chemical Stability Data

The Carbopol® formulation described in 2.1 was placed at 5° C. immediately after manufacturing and a couple of weeks later it was placed at 25° C. to initiate the stability. Then, it was thawed at 5° C., after which it was placed at 25° C. to initiate the stability. Chemical stability was assessed before the formulations were placed at 25° C., defined as the initial timepoint, and at 15 weeks. The following tests were performed to assess the chemical stability: the concentration of levodopa and carbidopa as percent from target (assay), and the key impurities (DHPA and DHPPA).

TABLE 4
Assay and Impurities of Formulations
on Stability at 25° C. over 15 weeks

| | | Carbopol ® | Carbopol ® |
|---|---|---|---|
| | | Formulation | Formulation |
| Test | Condition | without Foil Pouch | with Foil Pouch |
| Levodopa Assay | initial | 96.5 | 96.5 |
| Total (%) | | | |
| Levodopa Assay | 25° C.: | 99.9 | 97.1 |
| Total (%) | 15 weeks | | |
| Carbidopa Assay | initial | 93 | 93 |
| Total (%) | | | |
| Carbidopa Assay | 25° C.: | 92.4 | 92.6 |
| Total (%) | 15 weeks | | |
| DHPA (%) | initial | 0.3 | 0.3 |
| DHPA (%) | 25° C.: | 1.7 | 0.78 |
| | 15 weeks | | |
| DHPPA (%) | initial | 0.58 | 0.58 |
| DHPPA (%) | 25° C.: | 2.7 | 1.2 |
| | 15 weeks | | |
| Total | initial | 0.88 | 0.88 |
| Degradation (%) | | | |
| Total | 25° C.: | 4.5 | 2 |
| Degradation (%) | 15 weeks | | |

### 2.3—Physical Stability of Samples on Stability

The uniformity of dispensed content (UDC) method was used to assess the physical stability of the formulations. The UDC method obtains API concentration in the gel as it is dosed. This simulates what a patient would be receiving per every 5 g of gel delivered by the pump, and ensures that a patient will be receiving consistent amounts of drug throughout the consumption of one DDR. The test was performed at each time point throughout the 15 week stability. Particle size distributions of the APIs used for the study were within the particle size limits mentioned herein. Results are summarized in Tables 5 (levodopa) and 6 (carbidopa) below.

TABLE 5
Uniformity of Dispensed Content
of Levodopa Samples on Stability

| | | Carbopol ® | Carbopol ® |
|---|---|---|---|
| | | Formulation without | Formulation with |
| | | Foil Pouch | Foil Pouch |
| | | Initial Yield | Initial Yield |
| Fraction | Condition | Value = 6.9 Pa | Value = 6.9 Pa |
| 1 | Initial | 99.1 | 99.1 |
| 2 | | 97.6 | 97.6 |
| 3 | | 96.5 | 96.5 |
| 4 | | 96.1 | 96.1 |
| 5 | | 96.1 | 96.1 |
| 6 | | 95 | 95 |
| 7 | | 95.6 | 95.6 |
| 8 | | 95.3 | 95.3 |
| 9 | | 97.7 | 97.7 |
| 10 | | 96.4 | 96.4 |
| Acceptance Value | | 3 | 3 |
| (≤12) | | | |
| 1 | 25° C.: | 103.1 | 97.2 |
| 2 | 15 weeks | 101.3 | 98.8 |
| 3 | | 100.8 | 98.1 |
| 4 | | 99.6 | 97.4 |
| 5 | | 99.5 | 97 |
| 6 | | 99.9 | 96 |
| 7 | | 99.1 | 96.1 |
| 8 | | 98.3 | 95.8 |
| 9 | | 98.2 | 94.5 |
| 10 | | 97.3 | 93.3 |
| Acceptance Value | | 7.3 | 6 |
| (≤12) | | | |

TABLE 6
Uniformity of Dispensed Content
of Carbidopa Samples on Stability

| | | Carbopol ® |
|---|---|---|
| | | Formulation with |
| | | Foil Pouch |
| | | Initial Yield |
| Fraction | Condition | Value = 6.9 Pa |
| 1 | Initial | 99.1 |
| 2 | | 97.6 |
| 3 | | 96.5 |
| 4 | | 96.1 |
| 5 | | 96.1 |
| 6 | | 95 |
| 7 | | 95.6 |
| 8 | | 95.3 |
| 9 | | 97.7 |
| 10 | | 96.4 |
| Acceptance Value | | 3 |
| (≤15) | | |
| 1 | 25° C.: | 97.2 |
| 2 | 15 weeks | 98.8 |
| 3 | | 98.1 |
| 4 | | 97.4 |
| 5 | | 97 |
| 6 | | 96 |
| 7 | | 96.1 |
| 8 | | 95.8 |
| 9 | | 94.5 |
| 10 | | 93.3 |
| Acceptance Value | | 6 |
| (≤15) | | |

### Example 3—Therapeutic Effect of Pharmaceutical Composition in Mini-Pigs

High concentration (HC) L-dopa/carbidopa intestinal gel using sodium carboxymethylcellulose was compared with a low concentration (LC) L-dopa/carbidopa intestinal gel sodium carboxymethylcellulose, and a High concentration L-dopa/carbidopa intestinal gel using Carbopol® 974P and tested in the following manner: A group of four mini-pigs each was administered LC L-dopa/carbidopa intestinal gel, HC L-Dopa/carbidopa intestinal gel, or L-dopa/carbidopa in a Carbopol® 974P carrier in a cross-over study design. Each of four mini-pigs was administrated one formulation on the first day of a week, followed by 1-week washout period before the second formulation was administered. The third formulation was administered to the same mini-pigs after another week of washout period. Total Dose: 11.07 mg/kg levodopa dose over 6.5 hrs.; 20 mg/mL

### Groups: LC; HC; Carbopol® 974P

Bolus dose: 2.53 mg/kg over 30 min Bolus dose
Infusion dose: 8.54 mg/kg over 6 hrs.

Bolus infusion rate:

LC=0.253 mL/kg/hr (For example: 10 kg pig=2.53 mL/hr pump rate)

HC=0.127 mL/kg/hr (For example: 10 kg pig=1.27 mL/hr pump rate)

Carbopol® 974P=0.127 mL/kg/hr (For example: 10 kg pig=1.27 mL/hr pump rate)

6 hr infusion rate:

LC=0.071 mL/kg/hr (For example: 10 kg pig=0.71 mL/hr pump rate)

HC=0.0355 mL/kg/hr (For example: 10 kg pig=0.355 mL/hr pump rate)

Carbopol® 974P=0.0355 mL/kg/hr (For example: 10 kg pig=0.355 mL/hr pump rate). Plasma sampling Time points: 0.5, 1, 1.5, 2, 4, 6, 8, 9, 10, 12h

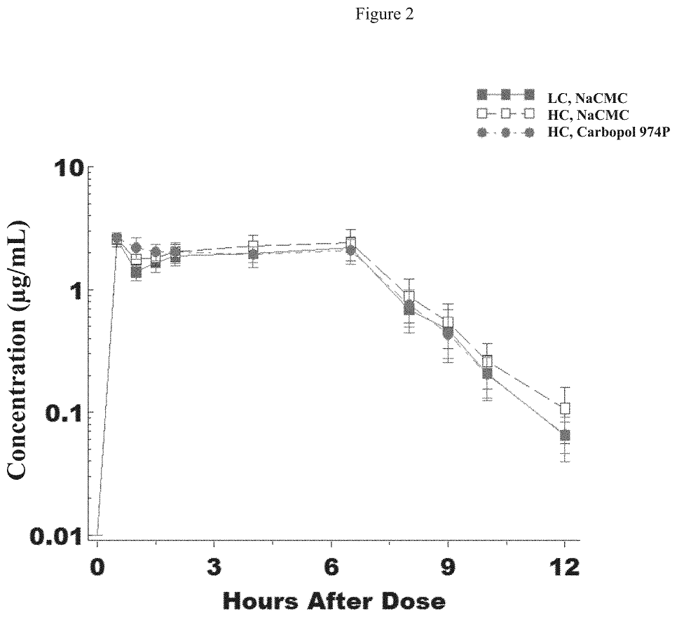

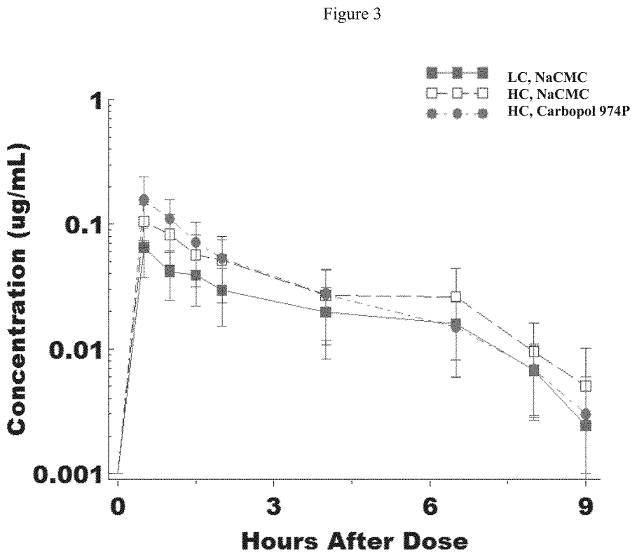

The results of the study confirm that the high concentration levodopa/carbidopa intestinal gel demonstrates comparable Cmax, Tmax and AUC values to the LC formulation when administered under half hour bolus and 6 hour infusion conditions, as shown in FIGS. 2 and 3. Cassettes of levodopa at 11.07 mg/kg and of carbidopa monohydrate at 2.77 mg/kg were dosed at 80 μL/kg (LC gel) or 40 μL/kg (HC gel and Carbopol® 974P control). Bioavailability of levodopa is summarized in Table 7 and 8 below. The half life value shown in Tables 7 and 8 are calculated as harmonic means.

TABLE 7
Levodopa plasma concentration

| Parameter | Mean (SEM) | | |
|---|---|---|---|
| Cₘₐₓ (μg/mL) | 2.69 | (0.26) | LC gel |
| Tₘₐₓ (h) | 2.0 | (1.5) | |
| AUC (μg · h/mL) | 15.9 | (3.35) | |
| t_{1/2} (h) | 1.1 | | |
| Cₘₐₓ (μg/mL) | 2.87 | (0.53) | HC gel |
| Tₘₐₓ (h) | 2.9 | (1.5) | |
| AUC (μg · h/mL) | 17.9 | (4.13) | |
| t_{1/2} (h) | 1.2 | | |
| Cₘₐₓ (μg/mL) | 2.75 | (0.31) | Carbopol ® 974P |
| Tₘₐₓ (h) | 2.1 | (1.5) | |
| AUC (μg · h/mL) | 16.4 | (2.97) | |

| | |
|---|---|
| t_{1/2} (h) | 1.0 |

Bioavailability of carbidopa is further summarized below.

TABLE 8
Carbidopa plasma concentration

| Parameter | Mean (SEM) | | |
|---|---|---|---|
| Cₘₐₓ (ng/mL) | 65 | (28) | LC gel |
| Tₘₐₓ (h) | 0.5 | (0) | |
| AUC (ng · h/mL) | 191 | (99) | |
| Cₘₐₓ (ng/mL) | 105 | (39) | HC gel |
| Tₘₐₓ (h) | 0.5 | (0) | |
| AUC (ng · h/mL) | 308 | (169) | |
| Cₘₐₓ (ng/mL) | 158 | (81) | Carbopol ® 974P |
| Tₘₐₓ (h) | 0.63 | (0.13) | |
| AUC (ng · h/mL) | 328 | (169) | |

The LC and HC L-dopa/carbidopa intestinal gels using sodium carboxymethylcellulose (NaCMC) were manufactured at the commercial scale. NaCMC was first added to water and was mixed at high shear using a homogenizer to prepare an aqueous gel. This gel was then sparged with nitrogen to remove most of the dissolved oxygen. Levodopa and carbidopa monohydrate were added to water in a separate vessel and was mixed at high shear using a homogenizer (this step can be performed using low shear) to make a slurry. The slurry was then added to the gel and was mixed at high shear using a homogenizer (the slurry step was repeated twice for the HC formulation). The entire composition was then degased and filled into cassettes. The HC formulation was then packaged in kits of 7 and immediately placed at −20° C. The LC formulation was packaged in foil pouches that were filled with an oxygen scavenger and were sparged with nitrogen before sealing. The HC L-dopa/carbidopa intestinal gel using Carbopol® 974P was prepared at lab scale using a process similar to that explained in Example 1.

### Example 4: Minimum Yield Value for Physical Stability at 12 Weeks, Room Temperature Using Carbopol® 971P

Three formulations of Levodopa-Carbidopa Intestinal Gel 40-10 mg/ml (LCIG 40/10) using 3 different concentrations of Carbopol® 971P: 0.1 w/w %, 0.2 w/w %, and 0.5 w/w % were prepared similar to Example 1. These were neutralized with NaOH so that the final pH was about 6.5. The ratio of Carbopol® to NaOH necessary to achieve a pH of about 6.5 was originally determined using the 0.2 w/w % Carbopol formulation.

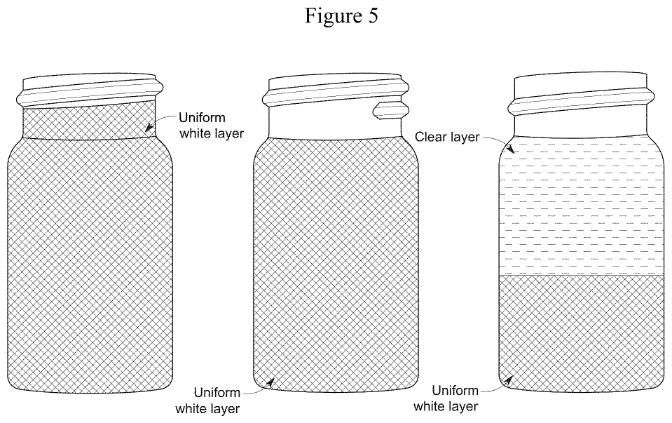

The 0.1 w/w %, 0.2 w/w %, and 0.5 w/w % formulations had yield values of 0.2 Pa, 1.1 Pa, and 4.5 Pa respectively. The formulations were placed in a tall vial at room temperature (about 20° C.) and were covered with cardboard to prevent degradation from light. All 3 formulations were uniformly white at the initial time point. After 8 weeks, the 0.1 w/w % formulation began to settle and a clear layer formed. After 12 weeks, the 0.5 w/w % and 0.2 w/w % formulations were still visually acceptable as shown in FIG. 5.

Based on these results, a yield value of at least 1.1 Pa is needed to resist sedimentation. The data is summarized below in Table 9.

TABLE 9
Minimum Yield Value Required to Resist Sedimentation

| | | Visual Observation |
|---|---|---|
| | Yield Value | After 12 Weeks |
| Formulation | at 25° C. | at about 20° C. |
| LCIG 40/10 0.5 w/w % | 4.5 Pa | Physically Stable |
| Carbopol ® 971P | | |
| LCIG 40/10 0.2 w/w % | 1.1 Pa | Physically Stable |
| Carbopol ® 971P | | |
| LCIG 40/10 0.1 w/w % | 0.2 Pa | Not Physically Stable |
| Carbopol ® 971P | | |

### Example 5: Physical Stability of Levodopa/Carbidopa Compositions

Pharmaceutical compositions comprising (a) levodopa in an amount of 3.88 w/w % of the total composition; (b) carbidopa monohydrate in an amount of 0.97 w/w % of the total composition; (c) a polymer-based suspending agent in the amount indicated; (d) sodium hydroxide in the amount indicated, and (e) water were prepared as outlined in Table 11. The UDC status in terms of levodopa acceptance value, viscosity, and yield value at various time points—week 1, week 8, and week 15—of these formulations are provided. The levodopa particle size (API particle size) is also provided. The compositions where stored at a temperature of about 25° C. and relative humidity of about 60% in a foil pouch. Based on these results, a yield value of at least 0.3 Pa is needed to resist sedimentation. The data are summarized in Table 11.

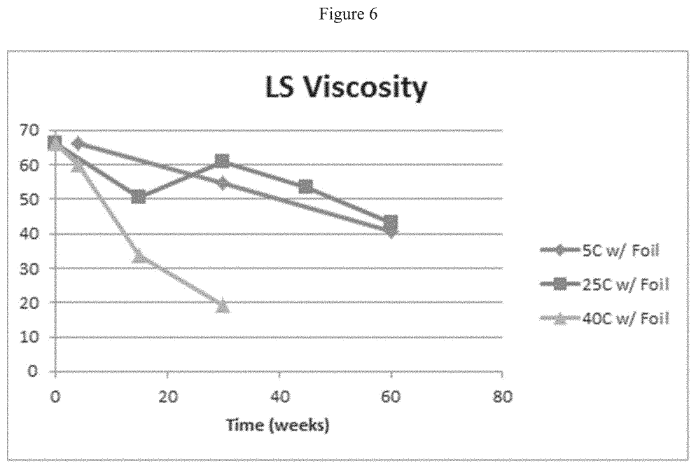

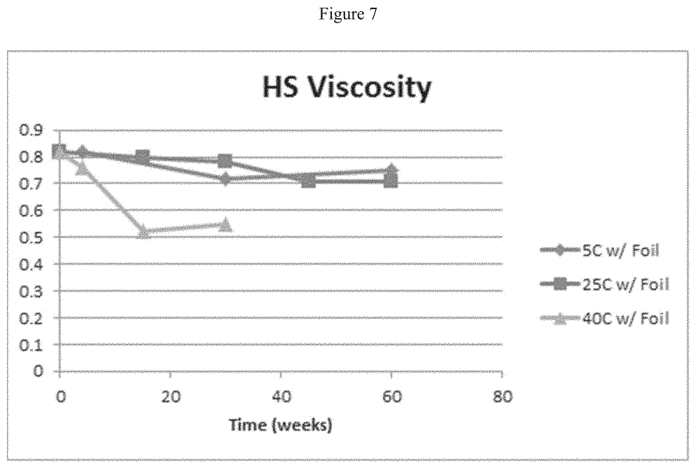

In a similar study, pharmaceutical compositions comprising (a) levodopa in an amount of 3.77 w/w % of the total composition (particle size: D50=3 μm, D90=7 μm and D100=18 μm); (b) carbidopa monohydrate in an amount of 0.94 w/w % of the total composition (particle size: (D50=2 μm, D90=6 μm and D100=16 μm); (c) Carbopol® 974P in an amount of 0.18 w/w % of the total composition; (d) sodium hydroxide in an amount of 0.306 w/w % of the total composition and (e) water were prepared and stored in a foil bag under the conditions indicated in Table 12. The physical stability and chemical stabilities of the pharmaceutical compositions are provided. The low shear (LS) viscosities measured at about 0.13 l/s and high shear (HS) viscosities measured at about 27 l/s of the formulations stored under the conditions indicated in Table 12 are shown in FIG. 6 and FIG. 7, respectively.

The yield values and viscosities in Table 11 and the yield values of Table 12 were measured using a Discovery HR-2 rheometer from TA instruments. The rheometer was fitted with a concentric cylinder. The sample was first soaked at 25° C. for 600 seconds, or until it reached equilibrium. The sample was presheared at 200 l/s for 120 seconds, if needed. An ascending flow-ramp was performed by first soaking the presheared sample for 360 seconds, then increasing the shear rate from 0.001 l/s to 250 l/s over 300 seconds. The sample was held at 250 l/s for an additional 300 seconds, if needed. Finally, a descending flow-ramp was performed from 250 l/s to 0.001 l/s over 300 seconds. The resulting descending flow-ramp were fitted with the Herschel-Bulkley model to obtain the yield value and viscosity.

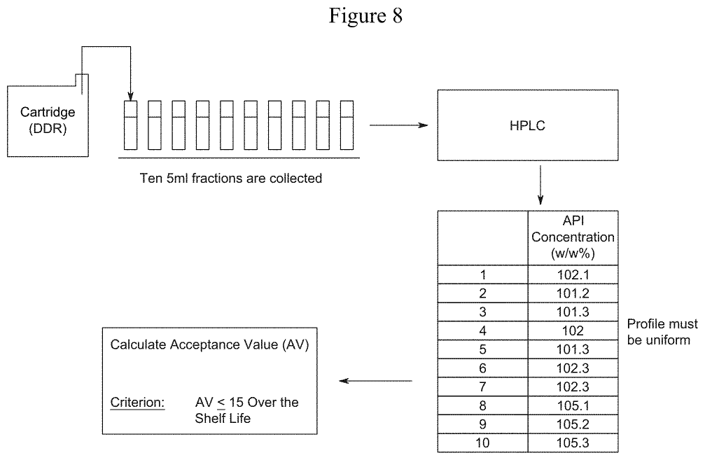

To determine the acceptance value, the pharmaceutical composition was withdrawn from a disposable drug reservoir (DDR) through integrated tubing in ten approximately equal fractions. Each portion was analyzed for drug concentration using High Pressure Liquid Chromatography. See USP, Chapter 905 and FIG. 8.

### Example 6: Mini-Pig Pharmacokinetic (PK) Study with Formulations Comprising Large and Small Levodopa Particle Sizes

Mini-pigs were surgically modified to place a J-tube into the jejunum a. Formulation

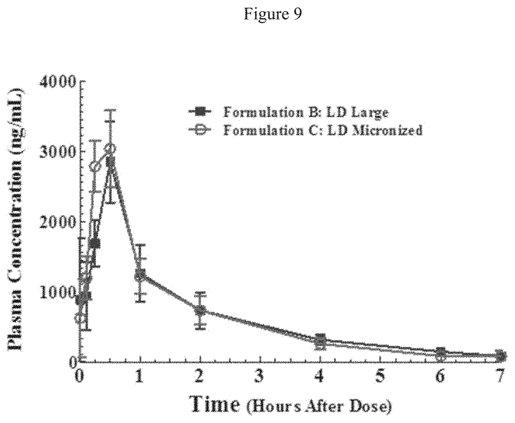

B (levodopa large particle size) and Formulation C (levodopa small particle size) were delivered through the J-tube via a medical pump. See Table 13. A crossover design was used with four mini-pigs in each arm. Each mini-pig was dosed 0.6 ml in 30 min at a constant infusion rate. Blood samples were withdrawn at 0, 0.1, 0.25, 0.5, 1. 2, 4, 6, 7 hrs, and plasma levodopa concentrations were measured by LC-MS. The PK data is shown in Table 14 and FIG. 9. There are no meaningful differences of AUC, Tmax, Cmax between the two gel formulations.

TABLE 13

| | Formulation B | Formulation C |
|---|---|---|
| | (w/w %) using | (w/w %) using |
| | large levodopa | small (micronized) |
| | particle size | levodopa particle |
| | about 37 μm (D50) | size ≤5 μm (D50) |
| Levodopa | 4 | 4 |
| Carbidopa Monohydrate | 1 | 1 |
| (particle size ≤3 μm) | | |
| Carbopol ® 974P | 0.18 | 0.18 |
| NaOH 18% w/w Solution | 0.31 | 0.31 |
| Water | 94.51 | 94.51 |
| Total | 100.00 | 100.00 |

TABLE 14

| | | Cₘₐₓ | Tₘₐₓ | AUC |
|---|---|---|---|---|
| | | (Mean | (Mean | (Mean |
| | t_{1/2} | and SEM) | and SEM) | and SEM) |
| | (Mean) | (ng/ml) | (*hr) | (ng*Hr/ml) |
| Formulation B | 1.4 (0.1) | 2850 (578) | 0.50 (0) | 4795 (1229) |
| Formulation C | 1.4 (0.1) | 3185 (432) | 0.38 (0.13) | 4868 (1198) |

TABLE 11

| | | | NaOH | Levodopa | UDC status | | | Viscosity | | | Yield value | | |
|---|---|---|---|---|---|---|---|---|---|---|---|---|---|
| | Suspending | | sol | Particle Size | (Levodopa AV value) | | | (Pa*s) | | | (Pa) | | |
| No | Agent | w/w % | (w/w %) | (D50) | Wk 0 | Wk 8 | Wk 15 | Wk 0 | Wk 8 | Wk 15 | Wk 0 | Wk 8 | Wk 15 |
| 1 | Carbopol ® 974P | 0.18% | 0.306 | about | Pass | Pass | Pass | 0.22 | 0.20 | 0.21 | 2.13 | 0.34 | 0.38 |
| | | | | 37.2 μm | (6.3) | (5.1) | (8.0) | | | | | | |
| 2 | Carbopol ® 974P | 0.15% | 0.255 | ≤5 μm | Pass | Pass | Pass | 0.11 | 0.08 | 0.11 | 2.88 | 1.34 | 2.33 |
| | | | | | (4.5) | (4.8) | (6.5) | | | | | | |
| 3 | Carbopol ® 974P | 0.12% | 0.255 | ≤5 μm | Pass | Pass | Fail | 0.12 | 0.06 | 0.05 | 3.44 | 0.65 | 0.27 |
| | | | | | (6.2) | (3.6) | (16.3) | | | | | | |
| 4 | Carbopol ® 974P | 0.09% | 0.255 | ≤5 μm | Fail | NA | NA | 0.03 | NA | NA | 0.08 | NA | NA |
| | | | | | (110.9) | | | | | | | | |
| 5 | Carbopol ® 934P | 0.15% | 0.255 | ≤5 μm | Pass | Fail | NA | 0.06 | 0.04 | NA | 0.03 | 0.008 | NA |
| | | | | | (3.7) | (62.4) | | | | | | | |
| 6 | Methylcellulose | 1.8% | none | ≤5 μm | Fail | NA | NA | 0.56 | NA | NA | 0.003 | NA | NA |
| | (1500 cps) | | | | (20.8) | | | | | | | | |
| 7 | Carbopol ® 934P | 0.3% | 0.51 | ≤5 μm | NA | NA | NA | 0.55 | NA | NA | 6.41 | NA | NA |
| 8 | Xanthan Gum | 1% | none | ≤5 μm | NA | NA | NA | 0.07 | NA | NA | 1.06 | NA | NA |
| | (Vanzan ®) | | | | | | | | | | | | |
| 9 | Avicel ®CL611 | 5% | none | ≤5 μm | NA | NA | NA | 0.14 | NA | NA | 0.83 | NA | NA |

TABLE 12

| | | Physical stability | Assay | | Hydrazine (μg/g) | | | | | |
|---|---|---|---|---|---|---|---|---|---|---|
| No | Conditions | (AV - levodopa) | (Levodopa) | | spec: NMT 40 | | DHPA (%) | | DHPPA(%) | |
| 1 | 5° C. | Initial AV = 6.6 | | | | | | | | |
| | | 30 weeks AV = 6.1 | 103.8% | (30 weeks) | | | 0.35 | (30 weeks) | 0.62 | (30 weeks) |
| | | 60 weeks AV = 3.8 | 95.6% | (60 weeks) | 6.6 | (60 weeks) | 0.44 | (60 weeks) | 0.72 | (60 weeks) |
| 2 | 25° C./60% RH | Initial AV = 3.0 | | | | | | | | |
| | | 15 weeks AV = 4.0 | 97.1% | (15 weeks) | 9.6 | (15 weeks) | 0.78 | (15 weeks) | 1.2 | (15 weeks) |
| | | 30 weeks AV = 3.4 | 96.3% | (30 weeks) | | | 1.3 | (30 weeks) | 2.0 | (30 weeks) |
| | | 45 weeks AV = 4.9 | 96.0% | (45 weeks) | | | 1.5 | (45 weeks) | 2.2 | (45 weeks) |
| | | 60 weeks AV = 4.3 | 95.8% | (60 weeks) | 16.3 | (60 weeks) | 1.4 | (60 weeks) | 1.9 | (60 weeks) |
| 3 | 40° C./75% RH | Initial AV = 6.6 | | | | | | | | |
| | | 4 weeks AV = 10.3 | 94.1% | (4 weeks) | 10.3 | (4 weeks) | 1.2 | (4 weeks) | 1.6 | (4 weeks) |
| | | 15 weeks AV = 15.1 | 95.5% | (15 weeks) | | | 2.3 | (15 weeks) | 2.6 | (15 weeks) |
| | | 26 weeks AV = 23.6 | 96.1% | (26 weeks) | 24.2 | (26 weeks) | 2.7 | (26 weeks) | 3.0 | (26 weeks) |

What is claimed is:

1. A pharmaceutical composition for intraduodenal administration comprising:
(a) a levodopa active agent in an amount of about 4.0 w/w % of the total composition;
(b) a carbidopa active agent in an amount of about 1.0 w/w % of the total composition;
(c) a polymer-based suspending agent in an amount of about 0.1 w/w % to about 5 w/w % of the total composition; and
(d) a liquid vehicle,
wherein:
(i) the liquid vehicle is water, polyethylene glycol, or a mixture of water and polyethylene glycol;
(ii) the acceptance value of the pharmaceutical composition is less than or equal to 15 with respect to the levodopa active agent and less than or equal to 15 with respect to the carbidopa active agent; and
(iii) the yield value of the pharmaceutical composition is at least about 0.3 Pa,
wherein:
the acceptance value and yield value are measured after exposing the pharmaceutical composition to a temperature of about 25° C. and relative humidity of about 60% for a period of at least about 8 weeks.

2. The pharmaceutical composition according to claim 1, wherein the polymer-based suspending agent is a carbomer.

3. The pharmaceutical composition according to claim 2, wherein the polymer-based suspending agent is Carbopol® 971P or Carbopol® 974P.

4. The pharmaceutical composition according to claim 2, wherein the composition comprises the polymer-based suspending agent in an amount of about 0.1 w/w % to about 0.3 w/w % of the total composition.

5. The pharmaceutical composition according to claim 1, wherein the polymer-based suspending agent is a hydrocolloid polymer.

6. The pharmaceutical composition according to claim 1, wherein the polymer-based suspending agent is selected from the group consisting of locust beam gum, guar gum, methylcellulose, sodium carboxymethylcellulose with microcrystalline cellulose, xanthan gum, and gum tragacanth.

7. The pharmaceutical composition according to claim 6, wherein the composition comprises the polymer-based suspending agent in an amount of about 1 w/w % to about 5 w/w % of the total composition.

8. The pharmaceutical composition according to claim 1, wherein the liquid vehicle is water.

9. The pharmaceutical composition according to claim 1 comprising:
(a) levodopa in an amount of about 4.0 w/w % of the total composition;
(b) carbidopa monohydrate in an amount of about 1.0 w/w % of the total composition;
(c) Carbopol® 971P or Carbopol® 974P in an amount of about 0.1 w/w % to about 0.2 w/w % of the total composition; and
(d) water.

10. The pharmaceutical composition according to claim 1, wherein the levodopa active agent has a median particle size distribution (D50) of ≤37 μm.

11. The pharmaceutical composition according to claim 1, wherein the carbidopa active agent has a median particle size distribution (D50) of about ≤10 μm.

12. The pharmaceutical composition according to claim 1, wherein the amount of impurities in the pharmaceutical composition is in an amount of less than about 5.8 w/w % of the total weight of the composition when maintained at a temperature of about 20-25° C. and a relative humidity of about 60% for a period of at least 15 weeks.

13. A pharmaceutical dosage form comprising the pharmaceutical composition according to claim 1 in a disposable drug reservoir having an oxygen impermeable enclosure disposed therein, wherein the oxygen impermeable enclosure is purged with an inert gas and an oxygen scavenger is added.

14. The pharmaceutical dosage form according to claim 13 comprising about 40 mg/mL of the levodopa active agent and about 10 mg/mL of the carbidopa active agent.

15. A pharmaceutical dosage comprising the pharmaceutical composition according to claim 9 in a disposable drug reservoir having an oxygen impermeable enclosure disposed therein, wherein the oxygen impermeable enclosure is purged with an inert gas and an oxygen scavenger is added.

16. A method of preparing the pharmaceutical composition comprising a levodopa active agent and a carbidopa active agent for intraduodenal administration, wherein
(i) the pharmaceutical composition has an acceptance value of the pharmaceutical composition is less than or equal to 15 with respect to the levodopa active agent and less than or equal to 15 with respect to the carbidopa active agent; and
(ii) the pharmaceutical composition has a yield value of at least about 0.3 Pa,
wherein:
the acceptance value and yield value are measured after exposing the pharmaceutical composition to a temperature of about 25° C. and relative humidity of about 60% for a period of at least about 8 weeks,
the method comprising:
adding an acrylic acid-based polymer suspending agent to water to form a dispersion;
adding a neutralizing agent to the dispersion to bring the pH to about 6.5 to form a medium;
adding a levodopa active agent and a carbidopa active agent to water to form a slurry; and
adding the slurry to the medium to form the pharmaceutical composition.

17. The pharmaceutical composition according to claim 16, wherein the acrylic acid-based polymer suspending agent is Carbopol® 971P or Carbopol® 974P.

18. A method of preparing the pharmaceutical composition comprising a levodopa active agent and a carbidopa active agent for intraduodenal administration, wherein
(i) the pharmaceutical composition has an acceptance value of the pharmaceutical composition is less than or equal to 15 with respect to the levodopa active agent and less than or equal to 15 with respect to the carbidopa active agent; and
(ii) the pharmaceutical composition has a yield value of at least about 0.3 Pa,
wherein:
the acceptance value and yield value are measured after exposing the pharmaceutical composition to a temperature of about 25° C. and relative humidity of about 60% for a period of at least about 8 weeks,
the method comprising:
adding a hydrocolloid polymer suspending agent to water to form a dispersion;
adding a levodopa active agent and a carbidopa active agent to water to form a slurry; and
adding the slurry to the medium to form the pharmaceutical composition.

19. The pharmaceutical composition according to claim 18, wherein the polymer-based suspending agent is selected from the group consisting of locust beam gum, guar gum, sodium carboxymethylcellulose with microcrystalline cellulose, xanthan gum, and gum tragacanth.

20. A method of treating Parkinson's disease in a patient in need thereof, the method comprising administering to the patient a therapeutically effective amount of the pharmaceutical composition according to claim 1.
